(19) United States
(12) Patent Application Publication
(10) Pub. No.: US 2026/0153496 A1
(43) Pub. Date: Jun. 4, 2026

(54) SENSING AN ANALYTE AND ASSESSING A HEMOLYSIS LEVEL IN BLOOD

(71) Applicant: Kalium Health Ltd., Girton, Cambridgeshire (GB)

(72) Inventors: Tanya HUTTER, Lakeway, TX (US); Thomas Stephen COLLINGS, Girton (GB); Gratsiela KOSTOVA, Cambridge (GB)

(73) Assignee: Kalium Health Ltd., Girton, Cambridgeshire (GB)

(21) Appl. No.: 18/998,976

(22) PCT Filed: Jul. 28, 2023
(86) PCT No.: PCT/GB2023/052012

§ 371 Date: Jan. 27, 2025

(30) Foreign Application Priority Data
Jul. 28, 2022 (GB) 2211014.2

(51) Int. Cl. G01N 33/49; G01N 21/25; G01N 21/64; G01N 27/333

(52) U.S. Cl. CPC G01N 33/491; G01N 21/251; G01N 27/333; G01N 21/64

(57) ABSTRACT

A sensor for sensing analytes in blood comprising plasma and erythrocytes, the sensor comprising: an analyte sensor configured to sense an analyte in the blood and to output a response corresponding to the sensed analyte; and a separation device adapted to mutually separate the plasma and the erythrocytes, for assessing a haemolysis level of the blood based on a colour and/or a fluorescence of the separated plasma.

### FIELD

The present invention relates to analysis of blood plasma or serum.

### BACKGROUND TO THE INVENTION

Conventionally, blood plasma or serum is analysed to measured concentrations of analytes for medical diagnosis.

Haemolysis is a major cause of blood sample invalidation in conventional blood testing (blood collected and subsequently tested at a later time). It has been reported to be responsible for 40% to 70% of unsuitable specimens leading to sample rejection. It has been reported that 3.3% of blood samples sent to biochemistry laboratories presented a hemolysis, of which more than 96% which presented haemolysis were due to in vitro mechanical haemolysis, mainly linked to the blood sampling material and shaking during pneumatic transport. Haemolysis is caused by the rupturing of erythrocytes (red blood cells, RBCs) resulting in release of intracellular components, such as high concentration of potassium (K) and haemoglobin (Hb) in plasma. The in vitro hemolysis leads to a release of hemoglobin (Hb), resulting in spectral interference during spectrophotometric measurements. Furthermore, other intrarerythrocytic components are released during in vitro hemolysis such as K or lactate dehydrogenase, consequently resulting in a false increase of their plasma or serum concentrations. Haemolysis results in increased levels of, for example, K in vitro being detected in drawn venous blood and incorrect diagnosis of patients, for example of patients having hyperkalaemia.

During a fingerstick blood draw for potassium detection, haemolysis assessment as quality control feature is highly desirable. Improper sample extraction may cause haemolysis of the sample. For example, applying pressure near the fingerstick puncture may cause rupture of RBCs, releasing potassium into the plasma, and resulting in false serum or plasma potassium assessment.

Currently, haemolysis levels can only be assessed after centrifugation of a venous draw sample (requiring benchtop technology and trained professionals). The haemolysis index (HI) is typically assessed based on the serum haemoglobin (free haemoglobin) concentration of the sample using a spectrophotometric analyser. Suitable spectrophotometric analysers include the Modular® analysers (Roche Diagnostics) such as Modular P800®. Another spectrophotometric analyser includes the Architect c16000 platform (Abbott Diagnostics). For the analyser to measure the HI, the samples must undergo dilution with saline solutions followed by polychromatic photometric detection of the interferent (Hb). These additional steps may add error and lab time to the analysis process and preclude Point of Care (POC) applications.

Hence, there is a need to improve analysis of blood.

### SUMMARY OF THE INVENTION

It is one aim of the present invention, amongst others, to provide a sensor which at least partially obviates or mitigates at least some of the disadvantages of the prior art, whether identified herein or elsewhere. For instance, it is an aim of embodiments of the invention to provide a sensor that senses analyte in blood and an assessment of a haemolysis level of the blood.

A first aspect provides a sensor for sensing analytes in blood comprising plasma and erythrocytes, the sensor comprising:
an analyte sensor configured to sense an analyte in the blood and to output a response corresponding to the sensed analyte; and
a separation device adapted to mutually separate the plasma and the erythrocytes, for assessing a haemolysis level of the blood based on a colour and/or a fluorescence of the separated plasma.

A second aspect provides an apparatus configured to receive a sensor according the first aspect, comprising:
an analyte detector configured to detect the response, corresponding to the sensed analyte, output from the analyte sensor; and
an optical detector configured to detect the colour and/or the fluorescence of the plasma for assessing the haemolysis level of the blood.

A third aspect provides a kit comprising a sensor according to the first aspect and an apparatus according to the second aspect.

A fourth aspect provides a method of sensing analytes in blood comprising plasma and erythrocytes, the method comprising:
mutually separating the plasma and the erythrocytes;
sensing an analyte in the blood and/or the plasma and outputting a response corresponding to the sensed analyte;
assessing a haemolysis level of the blood based on a colour and/or a fluorescence of the separated plasma; and
indicating a quality of the response corresponding to the sensed analyte based on the haemolysis level.

### DETAILED DESCRIPTION OF THE INVENTION

According to the present invention there is provided a sensor, as set forth in the appended claims. Also provided is an apparatus, a kit and a method. Other features of the invention will be apparent from the dependent claims, and the description that follows.

Sensor The first aspect provides a sensor for sensing analytes in blood comprising plasma and erythrocytes, the sensor comprising:
an analyte sensor configured to sense an analyte in the blood and to output a response corresponding to the sensed analyte; and
a separation device adapted to mutually separate the plasma and the erythrocytes, for assessing a haemolysis level of the blood based on a colour and/or a fluorescence of the separated plasma.

Thus, the response corresponding to the sensed analyte is output by the analyte sensor while the plasma and the erythrocytes are mutually separated, for assessment of the haemolysis level of the blood based on the colour and/or the fluorescence of the separated plasma. In this way, a quality (also known as confidence) of the response corresponding to the sensed analyte may be indicated qualitatively and/or quantitatively based on the assessed haemolysis level. For example, if the haemolysis level of the blood is relatively high, the response corresponding to the sensed analyte may be determined to be unreliable. Conversely, if the haemolysis level of the blood is relatively low, the response corresponding to the sensed analyte may be determined to be reliable. Since the sensor comprises the analyte sensor and the separation device, sensing of the analyte in the blood and assessment of the haemolysis level of the blood based on the colour and/or the fluorescence of the separated plasma may be simultaneous, thereby eliminating the conventional requirement for separate assessment of the haemolysis level of the blood. In this way, the sensor is particularly suitable for POC applications.

In one example, the analyte sensor and the separation device are fluidically coupled mutually in parallel, for example wherein the separation device is disposed above, below and/or beside (i.e. adjacent to) the analyte sensor. In this way, sensing of the analyte and mutual separation of the plasma and the erythrocytes are concurrent.

In one example, the analyte sensor and the separation device are fluidically coupled mutually in series. In this way, sensing of the analyte and mutual separation of the plasma and the erythrocytes are consecutive.

In one example, the analyte sensor and the separation device are fluidically coupled mutually in series and the analyte sensor is disposed in and/or on the separation device. In this way, the analyte sensor and the separation device are mutually integrated, as described below in more detail. In this way, the analyte sensor may be configured to sense the analyte in the separated plasma.

In one example, the analyte sensor and the separation device are fluidically coupled mutually in series and wherein the separation device is disposed downstream of the analyte sensor. In this way, the analyte sensor is configured to sense the analyte in the whole blood and the mutual separation of the plasma and the erythrocytes is downstream thereof.

In one example, the analyte sensor and the separation device are fluidically coupled mutually in series and wherein the separation device is disposed upstream of the analyte sensor. In this way, the analyte sensor is configured to sense the analyte in the mutually separated plasma or erythrocytes, preferably in the mutually separated plasma.

In one example, the separation device comprises and/or is an active separation device, for example based on bifurcation, constriction-expansion, biophysical and/or geometrical separation, and/or a passive separation device, for example based on a microbead plug, a porous microfilter, a membrane filter, blood agglutination, a nanofilter and/or superhydrophobicity.

The traditional method for separating plasma from whole blood in lab settings is centrifugation. Plasma separation in both traditional blood centrifuges and in lab-on-chip platforms require bulky equipment, which limits their application in the POC setting. Thanks to the development of microfluidic technology, it is now possible to separate plasma in small chips for micro total analysis. Microfluidic plasma separation methods include active separation and passive separation. For active separation methods, an external force/field is applied to the fluidic system, such as pumping, centrifugation or an acoustic, electric, or magnetic field. Passive separation requires no external forces and/or energy and is therefore better suited for application at the POC. Passive separation relies on the different behaviour of species (different cells and plasma) in the fluidic system. Different passive separation methods include sedimentation, filtration, lateral displacement, and hydrodynamic effects. Capillary flow-driven blood plasma separation is often used in lateral flow test strips because it is self-contained and autonomous and low-cost and comes with low fabrication and device complexity. For example, a filtration membrane may be integrated upstream on the lateral flowtest substrate. Plasma separation may be by red blood cell agglutination on paper-based microfluidic devices. Blood processing in high-surface-to-volume components, however, results in a significant loss of protein, typically more than 25% in plasma filtration membranes.

In one example, the separation device comprises and/or is a passive separation device based on a capillary bed for example a porous microfilter (also known as whole blood separator in membrane separation technology, blood cell separation membrane, plasma separation membrane or plasma separation pad), a microstructured polymer or a sintered polymer. Such capillary beds have the capacity to transport fluid (e.g., urine, blood, saliva) spontaneously.

Suitable porous microfilters are based on asymmetric polysulfone and asymmetric polyethersulfone, such as Pall Vivid Plasma Separation Membrane (asymmetric polysulfone) and Cobetter Plasma Separation Membrane (hydrophilic and highly asymmetric polyethersulfone), respectively. These porous microfilters provide physical retention, having asymmetric or highly asymmetric pore structures designed for whole blood plasma separation. Red blood cells and white blood cells are physically retained in relatively larger pores in the membrane while plasma conveys out via relatively smaller pores, separating the plasma from the red blood cells without haemolysis. In this way, the erythrocytes are retained upstream of the mutually separated plasma. It should be understood that generally, mutual separation of the plasma and the erythrocytes is not 100% efficient such that some residual plasma is retained also with the erythrocytes. However, typically few or no erythrocytes (i.e. a de minimis amount) are conveyed with the mutually separating plasma, such that assessing the haemolysis level of the blood based on the colour and/or the fluorescence of the separated plasma is not significantly affected thereby.

Pall's Vivid Plasma Separation membranes are robust materials for the rapid and efficient separation of plasma from whole blood. The membrane can separate >80% of the theoretical plasma available from the whole blood sample with minimal hemolysis in less than two minutes. Multiple grades of the membrane are available to support the blood volume requirements of your assay. The highly asymmetric nature of the Vivid Plasma Separation (PS) membrane allows efficient removal of the cellular components of blood without centrifugation. Red cells, white cells and platelets are captured within the larger pores on the upstream side of the membrane. Cells do not lyse, and the plasma flows through the smaller pores on the downstream side of the membrane. This rapid separation process yields plasma similar in HPLC and SDS-PAGE profiles to traditional centrifuged plasma. Hemolysis levels are significantly lower than glass fiber media-generated plasma. Vivid PS membrane can yield ≥80% of the theoretical plasma available, while comparable glass fibre yields are typically in the region of 30-50%. The percent of plasma recovered from different volumes of blood does not depend on the blood volume applied to the Vivid PS membrane. High plasma yields, mean a lower volume of starting whole blood is needed. This is advantageous for POC, and point of use (POU) diagnostic applications as smaller amounts of blood are needed from patients or animals. Whole blood processed through the Vivid PS membrane has shown equivalent 2D SDS-PAGE protein profiles for the cardiac biomarker Troponin I as compared to centrifuged plasma. Compatible with POC and POU diagnostic platforms such as lateral flow test strips and microfluidics.

Vivid membrane for separating plasma from whole blood is available in three grades designed for specific applications:

| Grade | Dimensions |
|---|---|
| GF | Small blood volume applications, such as finger |
| | sticks in microfluidic and lateral flow format POC |
| | devices. This material is untreated and may exhibit |
| | higher hemolysis levels than other grades. |
| GX | Small blood volume applications, such as finger |
| | sticks in microfluidic and lateral flow format POC |
| | devices. Also compatible with electrochemical analyte |
| | detection. Post-treatment helps to minimize hemolysis. |
| GR | Larger blood volume applications, such as lateral flow |
| | immunochromatographic devices. Post-treatment |
| | facilitates larger blood volumes with reduced hemolysis. |

Vivid membrane grade GX or equivalent or similar is preferred.

### Specifications

| | | | | Typical Performance Characteristics | | | |
|---|---|---|---|---|---|---|---|
| Typical Membrane Characteristics | | | | Blood | Membrane | Plasma | Plasma |
| Base | | Thickness | | Volume | Void | Separation | Recovery |
| Material | Grade | mils | μm | (μL/cm²) | Volume | Time (sec) | (%) |
| Aysmmetric | GF | 12.99 +/− | 330 +/− | 20 | 1X | ≤2 mins | ≤60% |
| Ploysulfone | | 0.79 | 20 | | | | |
| Aysmmetric | GX | 12.99 +/− | 330 +/− | 20-30 | 1.5X | ≤2 mins | ≤60% |
| Ploysulfone | | 0.79 | 20 | | | | |
| Aysmmetric | GR | 12.99 +/− | 330 +/− | 40-50 | 2X | ≤2 mins | ≤80% |
| Ploysulfone | | 0.79 | 20 | | | | |

Note:
The product performance characteristics outlined above are determined using EDTA collected whole blood with typical hematocrit content of 45.6%. For effective sample preparation it is important to ensure that the membrane lies flat, that no end contact occurs, and the edges are sealed by compression. In addition, the underlying material must have enough capillary force to wick the plasma from the Vivid PS membrane.
Note:
The sample blood volume capacity of Vivid PS membrane is defined as the amount of whole blood per cm² of medium that is separated in less than 2 minutes by the medium without hemolysis. The blood volume capacity of the medium is directly related to its void volume. The calculated void volume of Vivid PS membrane is ~20 ± 1 μ cm². With knowledge of the void volume within the Vivid PS membrane, and the specific membrane post-treatments utilized, we express recommended blood volume capacities that generate consistent plasma separation without hemolysis. These blood sample volume recommendations must be followed in order to achieve optimal plasma quality.

PSM series plasma separation membrane from Cobetter has a highly asymmetric pore structure, low haematocrit dependence, outstanding flow rate and separates plasma within two minutes. An anti-haemolysis treatment on the membrane reduces the possibility of whole blood haemolysis. The membrane has low non-specific binding. Plasma recovery is ≥10 μl @40 μl whole blood (recommended blood volume is 35 to 45 μl/cm². The membrane has a thickness of 0.34±0.03 mm.

Suitable porous microfilters are also based on cellulosic or glass fibre depth filters, such as Whatman® blood separators:

| | | | | Thickness | Wicking | Water |
|---|---|---|---|---|---|---|
| Volume | | | | (μm @ 53 | rate | absorption |
| per sq cm | | Grade | Materials and uses | kPA) | (s/4 cm) | (mg/cm²) |
| >50 | μl | VF2 | Vertical separator used as a single or | 785 | 23.8 | 86.2 |
| | | | multiple layers for separation. | | | |
| >50 | μl | GF/DVA | Untreated bound glass fiber. | 785 | 28.2 | 93 |
| 10-50 | μl | MF1 | Untreated bound glass fiber suitable for | 367 | 29.7 | 39.4 |
| | | | whole blood or serum. Typically used for | | | |
| | | | whole blood volumes between 30 μl | | | |
| | | | and 100 μl. | | | |
| 10-15 | μl | LF1 | Untreated bound glass fiber suitable for | 247 | 35.6 | 25.3 |
| | | | whole blood or serum. Performs well | | | |
| | | | with one or two drops of whole blood. | | | |
| 10-50 | μl | Fusion | Fusion 5 is a proprietary single-layer | 370 | 43.9 | 42.3 |
| | | 5 | matrix membrane that can perform all of | | | |
| | | | the functions of a lateral-flow strip. | | | |
| | | | It can be used as sample wick as well | | | |
| | | | as a blood separator with excellent | | | |
| | | | separation efficiency. | | | |

In one example, the capillary bed, such as a porous microfilter, a microstructured polymer or a sintered polymer, is elongate, having a symmetric shape such as a rectangle, a triangle or a hammer shape. In this way, the plasma is mutually separated from the erythrocytes by a relatively greater separation, improving assessing of the haemolysis level of the blood.

In one example, the capillary bed has a length in a range from 5 mm to 50 mm, preferably in a range from 10 mm to 25 mm, for example 15 mm and/or a width in a range from 1 mm to 10 mm, preferably in a range from 2 mm to 5 mm for example 2 mm or 3 mm.

In one example, the separation device is disposed for assessing the haemolysis level of the blood based on the colour and/or the fluorescence of the separated plasma measured optically, for example by absorbance, reflectance and/or transmission. In this way, the haemolysis level of the blood may be assessed based on the colour and/or the fluorescence of the separated plasma measured optically, for example by absorbance, reflectance and/or transmission, such as using spectrophotometry, colorimetry and/or fluorescence spectroscopy (also known as fluorimetry or spectrofluorometry).

Suitable devices for spectrophotometry and/or colorimetry of the plasma in and/or on the separation device include: a tristimulus colorimeter which measures the tristimulus values of a color; a spectroradiometer which measures the absolute spectral radiance (intensity) or irradiance of a light source; a spectrophotometer which measures the spectral reflectance, transmittance, or relative irradiance of a colour sample; a spectrocolorimeter which is a spectrophotometer that can calculate tristimulus values; a densitometer which measures the degree of light passing through or reflected by a subject; and a colour temperature meter measures the colour temperature of an incident illuminant. Additionally and/or alternatively, an image (such as a photograph or video) of the plasma in and/or on the separation device separation device may be acquired using an imaging device (such as a camera or video camera) and the colour of the separated plasma measured computationally. Suitable fluorescence spectrometers are known.

In one example, a surface, for example a single surface or opposed surfaces, of the separation device is exposed, for example directly or indirectly exposed such as to the ambient for example via an aperture and/or optically exposed for example via an optically transparent window. In this way, the haemolysis level of the blood may be assessed based on the colour and/or the fluorescence of the separated plasma measured optically, for example by absorbance, reflectance and/or transmission.

### Ion-Selective Electrode Cell

In one example, the analyte sensor comprises and/or is an ion-selective electrode cell, comprising an ion-selective electrode, a reference electrode and optionally, a counter electrode.

Generally, ion selective electrodes (ISEs) (also known as specific ion electrodes, SIEs), are transducers (also known as sensors) that convert ionic activities of selected ions in solutions into electrical responses, for example electrical potentials, currents and/or impedances. ISEs are used in ion-selective electrode cells, which include the ISEs in conjunction with reference electrodes. Concentrations of the selected ions in the solutions may be thus determined from the measured electrical responses, referenced to the reference electrodes. ISEs are used in analytical chemistry, environmental chemistry, food research, biomedical protocols and biochemical/biophysical research, typically for measurements of ionic concentrations in aqueous solutions.

The International Union of Pure and Applied Chemistry (IUPAC) Gold Book defines an ion-selective electrode (also known as a working electrode, WE) as an electrochemical sensor, based on thin films or selective membranes as recognition elements, and an electrochemical half-cell equivalent to other half-cells of the zeroth (inert metal in a redox electrolyte), 1st, 2nd and 3rd kinds. These devices are distinct from systems that involve redox reactions (electrodes of zeroth, 1st, 2nd and 3rd kinds), although they often contain a 2nd kind electrode as the ‘inner’ or ‘internal’ reference electrode. The potential difference response has, as its principal component, the Gibbs energy change associated with permselective mass transfer (by ion-exchange, solvent extraction or some other mechanism) across a phase boundary. The ion-selective electrode must be used in conjunction with a reference electrode (i.e. ‘outer’ or ‘external’ reference electrode) to form a complete electrochemical cell. The measured potential differences (ion-selective electrode vs. outer reference electrode potentials) are linearly dependent on the logarithm of the activity of a given ion in solution. Comment: the term ‘ion-specific electrode’ is not recommended. The term ‘specific’ implies that the electrode does not respond to additional ions. Since no electrode is truly specific for one ion, the term ‘ion-selective’ is recommended as more appropriate. ‘Selective ion-sensitive electrode’ is a little-used term to describe an ion-selective electrode. ‘Principal’ or ‘primary’ ions are those which an electrode is designed to measure. It is never certain that the ‘principal’ ion is most sensitively measured, e.g. nitrate ion-selective electrodes.

The IUPAC Gold Book defines an ion-selective electrode cell as an ion-selective electrode in conjunction with a reference electrode. Generally, the cell contains two reference electrodes, internal and external, and the thin film or membrane recognition-transduction element. However, besides this conventional type of cell (with solution contact on both sides of the membrane) there are cell arrangements with wire contact to one side of the membrane (all solid state and coated wire types).

It should be understood that the ISE is thus an ion-selective electrode and the RE is a reference electrode, according to these IUPAC Gold Book definitions.

There are four main types of ion-selective membrane used in ion-selective electrodes: glass, solid state, liquid-based, and compound electrode. In one example, the ISE comprises and/or is a glass membrane ISE, a solid-state ISE, a liquid-based ISE or a compound electrode ISE. Glass membranes are typically made from an ion-exchange type of glass (silicate or chalcogenide), though typically suitable for some single-charged cations such as H⁺, Na⁺, and Ag⁺. Chalcogenide glass also has selectivity for double-charged metal ions, such as Pb²⁺, and Cd²⁺.

Crystalline membranes are made from mono- or polycrystallites of a single substance and confer good selectivity on ISEs, because only ions that can introduce themselves into the crystal structure can interfere with the electrode response.

Ion-exchange resin membranes are based on organic polymer membranes which include a specific ion-exchange substance (resin). ISEs using ion-exchange resin membranes are in widespread use, including for analysis of anions. Usage of specific resins allows preparation of ISEs for tens of different ions, both single-atom or multi-atom. However, such ISEs tend to have low chemical and physical durability as well as ‘survival time’. Alkali metal ISE have been developed specifically for each alkali metal ion: Li⁺, Na⁺, K⁺, Rb⁺ and Cs⁺. The respective alkali metal ions are encapsulated in molecular cavities sized to match the ions. For example, a polymer-based membrane comprising an ionophore such as valinomycin or potassium ionophore Ill may be used for the determination of K⁺. Alkaline earth metal ISE have been developed specifically for each alkali metal ion: Be²⁺, Mg²⁺, Ca²⁺, Sr²⁺ and Ba²⁺. The respective alkaline metal ions are encapsulated in molecular cavities sized to match the ions. For example, a polymer-based membrane comprising an ionophore such as magnesium ionophore I may be used for the determination of Mg²⁺ or calcium ionophore IV for the determination of Ca²⁺ Enzyme electrodes are not true ion-selective electrodes but usually are considered within the ion-specific electrode topic. Such electrodes feature a double reaction mechanism in which an enzyme reacts with a specific substance, and the product of this reaction (usually H⁺ or OH⁻) is detected by a true ISE, such as a pH-selective electrode. An example is a glucose selective electrode.

In one example, the ISE comprises a membrane selected from a group comprising: a glass membrane, a crystalline membrane and an ion-exchange membrane, for example a polymer-based membrane.

In one example, the ion-selective electrode cell comprises the ISE and the RE provided on the separation device, such as a passive separation device based on a capillary bed such as a porous microfilter, a microstructured polymer or a sintered polymer (more generally, a substrate) as described below, and respective tracks for electrically coupling the ISE and the RE to a circuit, for example to a potentiometric circuit such as including a potentiometer and/or a galvanometric circuit (also known was an amperometric circuit), for example provided by the ISE. Hence, it should be understood that the ion-selective electrode cell does not include a potentiometric circuit such as including a potentiometer and/or a galvanometric circuit. In this way, a cost and/or a complexity of the ion-selective electrode cell may be reduced, such that the ion-selective electrode cell may be single-use (i.e. disposable) ion-selective electrode cell, electrically coupleable to a potentiometric circuit and/or a galvanometric circuit, for example provided by a device according to the fifth aspect. Two electrode ion-selective electrode cells (i.e. including the ISE and the RE) may be used for potentiometric measurements. In one example, the ion-selective electrode cell comprises a counter electrode (CE) (also known as an auxiliary electrode, AE). Three electrode ion-selective electrode cells (i.e. including the ISE, the RE and the CE) may be used for potentiometric and/or galvanometric measurements. In one example, the ion-selective electrode cell is a two electrode ion-selective electrode cell, comprising the ISE and the RE. In one example, the ion-selective electrode cell is a three electrode ion-selective electrode cell, comprising the ISE, the RE and a counter electrode, CE. In one example, the second electrode is the RE or the CE. In one preferred example, the ion-selective electrode cell is a three electrode ion-selective electrode cell, comprising the ISE, the RE and a counter electrode, CE, wherein the second electrode is the CE.

Generally, ion selective electrodes, ISEs, are transducers (also known as sensors) that convert ionic activities of specific ions in solutions into electrical responses, for example electrical potentials. The electrical potentials are theoretically dependent on the logarithms of the ionic activities, according to the Nernst equation:

$$E = {E}^{0} + \frac{RT}{{z}_{I} F} ln {a}_{I}$$

where
E is the expected electrical potential;
E⁰ is the standard electrical potential;
R is the universal gas constant;
T is the absolute temperature;
z_{I} is the charge on the ion (also known as ion of interest or primary ion);
F is Faraday's constant; and
a_{I} is the activity of the ion in the solution.

Hence, an ISE exhibits a Nernstian response if a ×10 change in the activity a_{I} of the ion results in approximately a 60 mV or a 30 mV change in the electrical potential E, for monovalent and for divalent ions respectively. In contrast, an ISE exhibits a super-Nernstian response when the ×10 change in the activity a_{I} of the ion results in a significantly larger change in the electrical potential E, for example exceeding 60 mV, 120 mV, 240 mV or even 700 mV for a monovalent ion.

Generally, the activity a_{I} of the ion is a measure of the ‘effective concentration’ of the ion in a mixture, in the sense that the ions ‘chemical potential depends on the activity of a real solution in the same way that it would depend on concentration for an ideal solution. However, a concentration of the ion is typically used in practice, rather than the activity a_{I} of the ion.

A polymer-based ISE typically comprises: an ionophore, to render selectivity to a membrane by forming a stable complex with the ion of interest; an ion-exchanger, to provide electroneutrality and ensure permselectivity; and a polymer matrix to provide support and mechanical functionality to the membrane. The polymer-based ISE response is now dictated by the phase boundary potential E_{PB}:

$${E}_{PB} = {E}^{0} + \frac{RT}{{z}_{I} F} ln \frac{{a}_{I , aq}}{{a}_{I , org}}$$

where:
a_{I,aq} is the activity of the ion in an aqueous phase; and
a_{I,org} is the activity of the ion in an organic phase.

In order to exhibit a Nernstian response, the activity of the ions in the bulk of the organic phase a_{I,org} must remain constant and independent of the sample. Therefore, the E_{PB} in such a case may be reduced to the Nernst equation:

$${E}_{PB} = {E}^{{0}^{′}} + \frac{RT}{{z}_{I} F} ln {a}_{I , aq}$$

In one example, the ion-selective electrode, the reference electrode and/or the counter electrode are disposed in and/or on the separation device.

In one example, the ISE and/or the RE is a screen-printed electrode (SPE), for example screen-printed on the onto the separation device, such as a passive separation device based on a capillary bed such as a porous microfilter, a microstructured polymer or a sintered polymer (more generally, a substrate). Generally, screen printing provides manufacture of SPEs in a reproducible, low-cost, and disposable format, while allowing ready incorporation of chemically functionalized materials. Screen-printing process has three main advantages over conventional methods of electrode manufacture: electrode area, electrode thickness, and electrode composition are readily controlled; statistical validation of experimental results is provided by replicate electrodes; and catalysts can be incorporated addition to screen-printing ink (paste). However, screen printing is generally restricted to planar substrates.

In one example, the ISE comprises and/or is formed, at least in part, from carbon, gold and/or platinum. Preferably, the ISE comprises and/or is formed, at least in part, from carbon, for example formed by screen-printing carbon ink onto the separation device, such as a passive separation device based on a capillary bed such as a porous microfilter, a microstructured polymer or a sintered polymer (more generally, a substrate).

In one example, the ISE comprises an ion-selective coating, for example overlaying carbon, gold and/or platinum. In one example, the ion-selective coating comprises a polymeric membrane providing a matrix, for example a neutral carrier-based solvent polymeric membrane such as based on plasticized poly(vinyl chloride) (PVC), polyurethane or a UV curable resin such as PU acrylate with acrylic monomer, comprising an ionophore, such as valinomycin, potassium ionophore Ill, magnesium ionophore I or calcium ionophore IV therein.

In one preferred example, the ISE comprises carbon and an ion-selective coating overlaying the carbon, wherein the ion-selective coating comprises a polymeric membrane providing a matrix, for example a neutral carrier-based solvent polymeric membrane such as based on plasticized poly(vinylchloride) (PVC), polyurethane or a UV curable resin such as PU acrylate with acrylic monomer, comprising an ionophore, such as valinomycin, potassium ionophore Ill, magnesium ionophore I or calcium ionophore IV therein.

In one example, the RE comprises and/or is a Ag or a Ag/AgCl reference electrode. In one preferred example, the RE is a Ag/AgCl reference electrode, for example, provided by screen-printing Ag/AgCl ink onto the separation device, such as a passive separation device based on a capillary bed such as a porous microfilter, a microstructured polymer or a sintered polymer (more generally, a substrate). In one example, the RE comprises and/or is a solid-state RE, for example based on doped conjugated and redox polymers, polymer composites and/or polymer electrolytes, such as derivatives of polypyrrole, polyamine and/or polythiophene. In one example, the RE comprises and/or is a poly(3,4-ethylenedioxythiophene) (PEDOT) doped with poly(styrenesulfonate) (PSS) reference electrode. Other REs are known, including metal and/or carbon REs, such as saturated calomel electrode, copper-copper (II) sulphate electrode, palladium-hydrogen electrode and mercury-mercurous sulfate electrode.

In one example, the ion-selective electrode cell comprises a counter electrode (CE). In one example, the CE comprises and/or is formed, at least in part, from carbon, gold and/or platinum, as described with respect to the ISE. In one preferred example, the CE comprises and/or is formed, at least in part, from carbon, for example, formed by screen-printing carbon ink onto a substrate, as described with respect to the ISE. In one example, the CE is uncoated (i.e. in contrast to the ISE).

### Analyte

In one example, the analyte is one or more selected from potassium (K), lactate dehydrogenase (LDH), aspartate aminotransferase (AST), alanine aminotransferase, magnesium, phosphate, folate, urea, glucose, sodium, chloride, bilirubin, gamma-glutamyltransferase, alkaline phosphatase, albumin, insulin, glucagon, calcitonin, parathyroid hormone, adrenocorticotropic hormone, gastrin, alkaline phosphatase (ALP), gamma-glutamyltransferase (GGT), bilirubin, creatine kinase (CK) and troponin T.

Generally, substances having plasma or serum concentrations at least ten times lower than in red blood cells are particularly susceptible to increases due to haemolysis. The three main mechanisms of interference from haemolysis are:
(i) additive interference of released intracellular substances added to plasma or serum measurement (e.g. LDH, AST, K);
(ii) spectral interference due to released haemoglobin (e.g. ALP, GGT); and
(iii) chemical interference when intracellular substances interact with the measured analyte (e.g. CK).

Accordingly, plasma levels of potassium (K), lactate dehydrogenase (LDH), aspartate aminotransferase (AST), alanine aminotransferase, magnesium, phosphate, folate and urea concentrations may be falsely increased. Variations in K results are particularly important since they may incorrectly suggest a life-threatening situation and so lead physicians to make inappropriate treatment changes. Red blood cells also contain high amounts of neuron-specific enolase (NSE), resulting in falsely elevated NSE concentrations measured in hemolyzed serum or cerebrospinal fluid and complicating the diagnosis of small-cell lung cancer and neuroblastoma. Levels of blood components with a mainly extracellular localisation may be falsely decreased in case of sample haemolysis as a result of plasma or serum dilution due to release of intraerythrocytic fluid. This is the case for glucose, sodium, chloride, bilirubin, gamma-glutamyltransferase, alkaline phosphatase and albumin.

Release of proteolytic enzymes from the red blood cells causes degradation of insulin, glucagon, calcitonin, parathyroid hormone, adrenocorticotropic hormone and gastrin. Therefore, the measured concentration of these hormones may be decreased. Hb released from red blood cells absorbs visible light at wavelengths of mainly 415 nm, 540 nm and 570 nm, causing interference with spectrophotometric measurements at these wavelengths. Levels of alkaline phosphatase, gamma-glutamyltransferase, and bilirubin may be falsely decreased when measured spectrophotometrically, whilst lipase and iron levels may be falsely increased.

Adenylate kinase, when released from erythrocytes, causes an increase in measured creatine kinase concentration. The release of Hb from red blood cells also causes a positive interference in the troponin I assay, whilst it induces false negative results for troponin T along with the release of proteolytic enzymes. Interference of haptoglobin-Hb complexes and free Hb with serum protein electrophoresis is possible. Finally, antibodies used in immunological tests can cross-react with components released from the red blood cells. The unpredictable response of several blood components to haemolysis precludes the implementation of usable correction factors.

### Device

In one example, the sensor is provided in the form of a point of care (POC) or point of use (POU) diagnostic platform such as a lateral flow test strip and or microfluidic test strip, for single use.

### Apparatus

The second aspect provides an apparatus configured to receive a sensor according the first aspect, comprising:
an analyte detector configured to detect the response, corresponding to the sensed analyte, output from the analyte sensor; and
an optical detector configured to detect the colour and/or the fluorescence of the separated plasma for assessing the haemolysis level of the blood.

Suitable analyte detectors are known.

In one example, the analyte detector comprises a circuit, for example a potentiometric circuit such as including a potentiometer circuit and/or a galvanometric circuit for electrically coupling to an ion-selective electrode cell, comprising an ion-selective electrode, a reference electrode and optionally, a counter electrode, wherein the analyte sensor comprises and/or is the ion-selective electrode cell.

In one example, the optical detector comprises:
a light source arranged to illuminate the separated plasma; and
a light sensor arranged to sense light absorbed by, reflected by and/or transmitted through the separated plasma;
optionally one or more spectral filters (for example, to provide a band pass filter targeted on a particular wavelength or range of wavelengths) and/or one or more lenses and/or one or more mirrors disposed between the light source and the separation device;
optionally one or more spectral filters (for example, to provide a band pass filter targeted on a particular wavelength or range of wavelengths) and/or one or more lenses and/or one or more mirrors disposed between the separation device and the light sensor.

In one example, the light source comprises and/or is ambient lighting, a lamp or a light emitting diode (LED), preferably a LED. LEDs are relatively low cost, of relatively low complexity and are relatively small. In one example, the light source is of one or more selected wavelengths and/or one or more selected wavelength ranges.

In one example, the light sensor comprises and/or is a photodetector or photodiode, an image sensor such as a charge-coupled detector (CCD) image sensor, a complementary metal-oxide semiconductor (CMOS) image sensor, a camera, a video camera, preferably a photodetector or photodiode. Photodetectors or photodiodes are relatively low cost, of relatively low complexity and are relatively small. In one example, the light sensor is for one or more selected wavelengths and/or one or more selected wavelength ranges. In one example, the one or more selected wavelengths include one or more of 415 nm, 540 nm and 570 nm. In one example, the one or more selected wavelength ranges include one or more of 415 nm, 540 nm and 570 nm. Hb released from red blood cells absorbs visible light at wavelengths of mainly 415 nm, 540 nm and 570 nm.

In one example, the optical detector is configured to detect the colour and/or the fluorescence of the separated plasma flow front. In this way, the optical detector detects a colour change and/or a fluorescence change of the separation device due to the separated plasma. In one example, the optical detector is configured to detect the colour and/or the fluorescence of the separated erythrocytes. In one example, the optical detector is configured to discriminate the colour and/or the fluorescence of the separated plasma and the colour and/or the fluorescence of the separated erythrocytes. In this way, the optical detector can detect if the separated erythrocytes are detected rather than the separated plasma. For example, if the blood sample volume is excessive, the separated erythrocytes flow front may move beyond a region detected by the optical detector such that the separated erythrocytes are detected.

In one example, the apparatus is configured to output a value corresponding with the haemolysis level of the blood based on the detected colour and/or the detected fluorescence of the separated plasma, as described with respect to the first aspect.

In one example, the value comprises and/or is a ratio of two or more wavelengths of the light reflected by and/or transmitted through the separated plasma, as described with respect to the first aspect.

In one example, the apparatus is configured to estimate the value corresponding with the haemolysis level of the blood based on the detected colour and/or the detected fluorescence of the separated plasma using a trained machine learning algorithm, for example wherein the machine learning algorithm is trained using supervised learning using samples of haemolysed and non-haemolysed blood, for example having different haemolysis levels. Methods of synthesising haemolysed blood are known.

In one example, the apparatus is configured to implement a remedial action based on the value, for example wherein the value is greater than a predetermined upper threshold, less than a predetermined lower threshold and/or outside or inside a predetermined range. In this way, the remedial action may be implemented if the value indicates that the blood is haemolysed.

In one example, the remedial action is selected from: displaying a message for example via a GUI, transmitting a message, illuminating a light, sounding an audible alarm, flagging the value, repeating a measurement.

In one example, the apparatus is configured to correct the response, corresponding to the sensed analyte, based on the haemolysis level of the blood, for example using the value corresponding with the haemolysis level of the blood. In this way, the detected response, corresponding to the sensed analyte, may be corrected for haemolysis of the blood.

### Kit

The third aspect provides a kit comprising a sensor according to the first aspect and an apparatus according to the second aspect.

### Method

The fourth aspect provides a method of sensing analytes in blood comprising plasma and erythrocytes, the method comprising:
mutually separating the plasma and the erythrocytes;
sensing an analyte in the blood and/or the plasma and outputting a response corresponding to the sensed analyte;
assessing a haemolysis level of the blood based on a colour and/or a fluorescence of the separated plasma; and
indicating a quality of the response corresponding to the sensed analyte based on the haemolysis level.

In one example, the method comprises correcting the response, corresponding to the sensed analyte, based on the haemolysis level of the blood. In this way, the detected response, corresponding to the sensed analyte, may be corrected for haemolysis of the blood.

### Definitions

Throughout this specification, the term “comprising” or “comprises” means including the component(s) specified but not to the exclusion of the presence of other components. The term “consisting essentially of” or “consists essentially of” means including the components specified but excluding other components except for materials present as impurities, unavoidable materials present as a result of processes used to provide the components, and components added for a purpose other than achieving the technical effect of the invention, such as colourants, and the like.

The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

### BRIEF DESCRIPTION OF THE DRAWINGS

For a better understanding of the invention, and to show how exemplary embodiments of the same may be brought into effect, reference will be made, by way of example only, to the accompanying diagrammatic Figures, in which:

FIG. 1A summarises results of fHb concentration and RGB ratios for seven samples 1 to 7 (replicates n=3), for non-haemolysed blood, slightly haemolysed and haemolysed blood having relatively higher haemolysis levels; FIG. 1B is a graph of RGB ratio as a function of fHb concentration for the five samples; FIG. 1C is a graph of RGB ratio as a function of fHb concentration for the five samples 1 to 5 (excluding samples 6 and 7 having haemolysed plasma too saturated for fHb calculation for which dilution is required but graph would not be a straight line), showing straight line of best fit for the linear relationship between RGB ratio and fHb concentration; and FIG. 1D shows raw spectra of extracted plasma samples 1 to 5 between 190 to 1100 nm measured using a NeoDot UVVis Nano Spectrophotometer.

FIG. 2 schematically depicts seven sensors (1 to 7) according to exemplary embodiments;

FIGS. 3A to 3G show open circuit potential (OCP) graphs of potential (V) as a function of time for the seven sensors (1 to 7), respectively, of FIG. 2, together with representative images of the separated blood and RGB values of the separated plasma, for potassium measurements of fingerstick blood using an ion-selective electrode, in conjunction to simultaneous haemolysis measurement on filter;

FIG. 4A schematically depicts an ion-selective electrode cell for a sensor according to an exemplary embodiment; and FIG. 4B schematically depicts an ion-selective electrode cell for a sensor according to an exemplary embodiment;

FIG. 5A schematically depicts an exploded, perspective view of a sensor according to an exemplary embodiment; and FIG. 5B schematically depicts an exploded, perspective view of a sensor according to an exemplary embodiment;

FIG. 6 schematically depicts a method according to an exemplary embodiment;

FIG. 7 is a photograph of an apparatus according to an exemplary embodiment;

FIG. 8A is a plan view from above of a sensor according to an exemplary embodiment; and FIG. 8B is a plan view from below of the sensor;

FIG. 9 is a photograph from above of a sensor according to an exemplary embodiment;

FIG. 10A is a graph of OCP response against time for each blood sample, recorded alongside the haemolysis detection device (Measurement begins before blood is applied. As blood is applied, spikes in response may be observed. The evolution of peaks in the curve is due to equilibration. OCP value for each curve recorded once response is stable); FIG. 10B shows images taken from videos recorded during the simultaneous potassium detection measurement and filter filling with blood—these images were analysed to produce the RGB values displayed; and FIG. 10C shows images from videos recorded during sensor and filter filling with blood—OCP was recorded simultaneously;

FIG. 11A is a graph of OCP response against time for each blood sample, recorded alongside the haemolysis detection device; FIG. 11B shows images taken from videos recorded during the simultaneous potassium detection measurement and filter filling with blood—these images were analysed to produce the RGB values displayed; and FIG. 11C shows images from videos recorded during sensor and filter filling with blood—OCP was recorded simultaneously;

FIG. 12A is a photograph from above of a sensor according to an exemplary embodiment; and FIG. 12B is a graph of absorbance as a function of wavelength showing raw spectra of extracted plasma samples 1 to 5 (decreasing haemolysis levels) between 190-1100 nm measured using NeoDot UVVis Nano Spectrophotometer;

FIGS. 13A to 13C are 3D printed structure of an apparatus according to an exemplary embodiment, showing LED and photoresistor positions;

FIG. 14 is a sectional view of the 3D printed structure of FIGS. 13A to 13C, secured by a clamp stand of the device and the installation positions of the LED and photoresistor, in more detail;

FIG. 15A is a graph of chronoamperometric response of LDR-filter interactions recorded for sample 5.3, overlaying a photograph of the filter of the sample 5.3; and FIG. 15B is a graph of chronoamperometric response of full set of samples 1 to 6 (3 repeats each); and

FIG. 16A is a graph of current change as a function of fHb for the full set of samples 1 to 6 (3 repeats each); and FIG. 16B is a graph of current change as a function of fHb for the full set of samples 1 to 6 (3 repeats each), excluding those samples in the non-linear region.

### DETAILED DESCRIPTION OF THE DRAWINGS

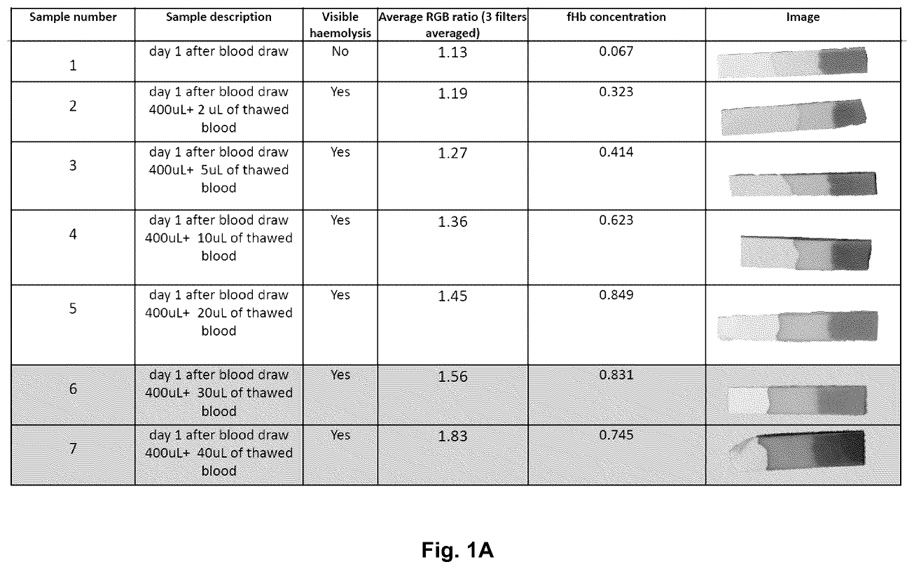

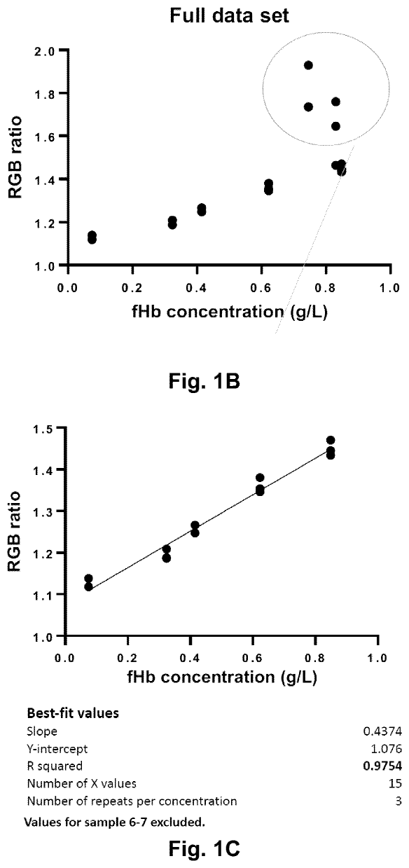

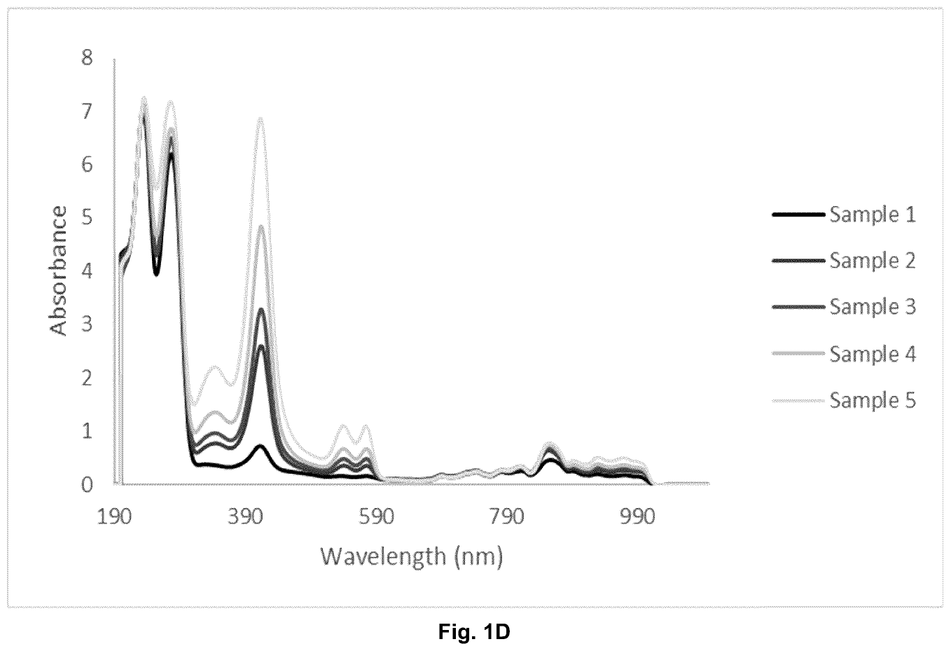

FIG. 1A summarises results of fHb concentration and RGB ratios for seven samples 1 to 7 (replicates n=3), for non-haemolysed blood and haemolysed blood having relatively higher haemolysis levels; FIG. 1B is a graph of RGB ratio as a function of fHb concentration for the five samples; FIG. 1C is a graph of RGB ratio as a function of fHb concentration for the five samples 1 to 5 (excluding samples 6 and 7 having haemolysed plasma too saturated for fHb calculation for which dilution is required but graph would not be a straight line), showing straight line of best fit for the linear relationship between RGB ratio and fHb concentration; and FIG. 1D shows raw spectra of extracted plasma samples 1 to 5 between 190 to 1100 nm measured using a NeoDot UV/Vis Nano Spectrophotometer.

The novel method suggested in this paper utilises a simple visual monitoring protocol of application of blood on filter, which laterally separates red blood cells from plasma. The plasma colour can then be analysed visually for qualitative assessment, or via colorimetric analysis for quantitative assessment of haemolysis. This colorimetric assessment can be done either in reflection or transmission modes using a spectrometer to gain full spectrum, via LEDs and photodetectors for narrower wavelength bands or via image/photo analysis. In this work, image analysis was done to determine the red index, green index and blue index and an equation for RGB ratio was proposed. Moreover, this method is compatible with the small volume of finger prick blood. In addition, the method can be integrated with disposable sensors for potassium and other analytes that are prone to haemolysis interference.

The method described relies on a filter paper which can cover three factors to perform the role required for our intended purpose. Firstly, the filter preferably separates RBCs from plasma laterally. Secondly, the colour of the separated plasma is preferably easy to observe on the surface of the filter. Thirdly, the filter paper preferably separates RBCs from plasma without causing lysis of cells. One filter which fits the purposes of investigation is the Vivid™ Plasma Separation Membrane by Pall. Guo et al. (2020) reported a plasma separator from whole blood by agglutination in the lateral flow test substrate synthetic paper. Tang et al. (2016) reviewed advances in paper-based pre-treatments in POC testing, such as blood separation membranes like LF1 by Whatman (previously trialed by Kalium). Li et al. (2019) further review blood separation methods and blood sampling methods on paper and the potential detection of markers such as haematocrit, glucose and blood grouping¹⁷.

### Materials and Methods

### Materials

Vivid™ Plasma Separation Membrane (Pall) was used as a lateral separator of plasma from whole blood. Potassium ion-selective sensors used were created by Flex Medical and functionalised by Kalium Health. Capillary channels were prepared by Quick Axis for Kalium Health. Venous blood was obtained from volunteer and a lithium-heparin S-Monovette 4.9 mL was used to store venous blood. The blood was centrifuged using an Eppendorf 5415 D. Fingerpick blood obtained from scientist performing experiments using a Haemolance plus 21 G 1.8 mm lancet. Potentiostat used was the Sensit Smart smartphone potentiostat. Spectrophotometer used was the NeoDot UV/Vis Nano Spectrophotometer (Generon).

### Methods

### Blood Collection

Venous blood was drawn from volunteer and stored in a lithium-heparin S-Monovette 4.9 mL tube overnight. A small volume of venous blood one day post draw was frozen for 45 minutes. The frozen blood was thawed to room temperature (fully haemolysed blood) and added to venous blood one day post draw kept at room temperature (non haemolysed blood) in ratios as described in Table S1.

TABLE S1
Mixing ratios of sample 1-5 of non-haemolysed blood (one
day old kept at room temperature) and fully haemolysed blood
(frozen for 45 minutes and thawed to room temperature.

| Sample | Non-haemolysed | Fully haemolysed |
|---|---|---|
| number | blood (μL) | blood (μL) |
| 1 | 400 | 0 |
| 2 | 400 | 2 |
| 3 | 400 | 5 |
| 4 | 400 | 10 |
| 5 | 400 | 20 |

### Standard fHb Method

The remaining samples left were spun down using an Eppendorf 5415 D centrifuge at 1000×g for 5 minutes. Following centrifugation, the plasma was extracted and spectra (wavelength 190-1100 nm) of 1.2 μL volume was collected using the NeoDot UV/Vis Nano Spectrophotometer of the five samples (n=1). An ultra-pure water sample was used as blank. The respective absorbances at 380 nm, 415 nm and 450 nm were measured and fHb was calculated using Equation (1).

### Haemolysis Filter Detection Method

The samples prepared were applied (4 μL volume) on the ends of rectangular Vivid™ Plasma Separation Membrane grade GX filter papers (cut into 2-3 mm by 15 mm strips by hand). Separation of plasma and red blood cells was observed. The colour of the separated plasma on the filters were recorded using a camera (Samsung Galaxy Tab A7) to take a photograph. Three repeats for each sample were carried out.

### Filter Embedding

The blood separating filter was embedded onto the Kalium Health sensors in seven different configurations. The Open Circuit Potential (OCP) between the ion-selective working electrode and the reference electrode was recorded over 90 seconds, t interval=0.2V. A Sensit Smart smartphone potentiostat on a Samsung Galaxy Tab A7 was used to obtain a measurement of the potassium response of the sensor, whilst simultaneously recording video of the filter separating the red blood cells from plasma (using an iPhone 11 camera).

### Results and Discussion

### Assessment of New Method of Haemolysis Filter Detection

The five blood samples described in Table S1 were assessed for haemolysis levels in two ways: using the standard fHb method and using the novel RGB method.

Firstly, in order to assess fHb concentration using the standard method, the spectra of the five samples were used to calculate the value using Equation (1) using the middle region of the filters. The data set was displayed in FIG. 1D and processed in Table S2.

TABLE S2
Processed results from the full range spectra of samples 1 to 5.

| | | | | fHb |
|---|---|---|---|---|
| Sample | Absorption | Absorption | Absorption | concentration |
| number | at 380 nm | at 415 nm | at 450 nm | (g/L) |
| 1 | 0.3896 | 0.7798 | 0.2852 | 0.0739613 |
| 2 | 0.8115 | 2.5849 | 0.4894 | 0.3234415 |
| 3 | 0.9706 | 3.2751 | 0.6219 | 0.4144663 |
| 4 | 1.3769 | 4.8342 | 0.8425 | 0.6227335 |
| 5 | 2.224 | 6.8614 | 1.3484 | 0.8485712 |

Assessment for free haemoglobin concentration (fHb) using a spectrophotometer was previously used in order to assess haemolysis of samples and is also recognised as a standard method of detection. Equation (1) represents the relationship between the fHb and absorbance used herein.

$$Equation ( 1 )$$

$$fHb ( {gL}^{- 1} ) = 83.6 \times \frac{( 2 \times [ Abat 415 nm ] - [ Abat 380 nm ] - [ Abat 450 nm ] )}{1000}$$

Secondly, the photographs of the filters (n=3) were also analysed using the novel RGB ratio method shown in Equation 2. The data are summarised in Table S3.

$$PlasmaRGBratio = \frac{Red}{AVG ( Blue + Green )} \qquad Equation ( 2 )$$

$$PlasmaRGBratio = \frac{Red}{Blue} \qquad Equation ( 3 )$$

$$PlasmaRGBratio = \frac{Red}{Green} \qquad Equation ( 4 )$$

TABLE S3
Table of RGB values measured from photographs of samples 1 to
5 (n = 3) and the RGB ratio calculated using Equation (2).

| Sample | | | | RGB Ratio |
|---|---|---|---|---|
| number | RED | GREEN | BLUE | Equation (2) |
| 1 | 185 | 171 | 160 | 1.12 |
| 1 | 187 | 173 | 162 | 1.12 |
| 1 | 186 | 170 | 157 | 1.14 |
| 2 | 204 | 181 | 163 | 1.19 |
| 2 | 203 | 180 | 162 | 1.19 |
| 2 | 212 | 185 | 166 | 1.21 |
| 3 | 212 | 180 | 155 | 1.27 |
| 3 | 212 | 182 | 158 | 1.25 |
| 3 | 209 | 178 | 157 | 1.25 |
| 4 | 207 | 160 | 140 | 1.38 |
| 4 | 207 | 165 | 141 | 1.35 |
| 4 | 220 | 175 | 152 | 1.35 |
| 5 | 219 | 161 | 137 | 1.47 |
| 5 | 230 | 174 | 147 | 1.43 |
| 5 | 229 | 172 | 145 | 1.44 |

This method evaluates how red a selected colour (such as image of plasma separated) appears. Non-haemolysed blood has plasma which appears yellow to clear. As blood become more haemolysed, the plasma colour appears redder.

It is important to note that the suggested Equation 2 is only one way of analysing the image of the blood in the filter. Other equations which may provide similar outcome also include Equation 3 and Equation 4, although their validity is not analysed in this report. Other calculations of simply the redness of the image will work too. So as other methods that include full UV-VIS spectral measurement and LEDs with photodetectors for partial spectral measurement.

The fHb concentration was plotted against the RGB ratio measurements in order to evaluate the validity of the method and the data (FIG. 1C).

A y-intercept of 1.076 was reported, favourably close in value to the expected y-intercept of 1.00. Linear regression analysis provides an R² value of 0.975 which increases the confidence in the method described. Overall, the RGB ratio maps on well to the standard method of haemolysis detection.

The RGB ratio values proved to be consistent in various lighting, brightness and cameras used in the preliminary evaluation of this experiment, proving the robustness of this method further. The superiority of the haemolysis filter detection method arises due to the cheap equipment required (camera vs spectrophotometer), the lack of centrifugation and therefore the ability to embed filter alongside a sensor strip and the low volume of blood required in order to perform the quality control. The filter size can be scaled in order to increase or decrease the required volume of blood in order to suit the needs of the design best.

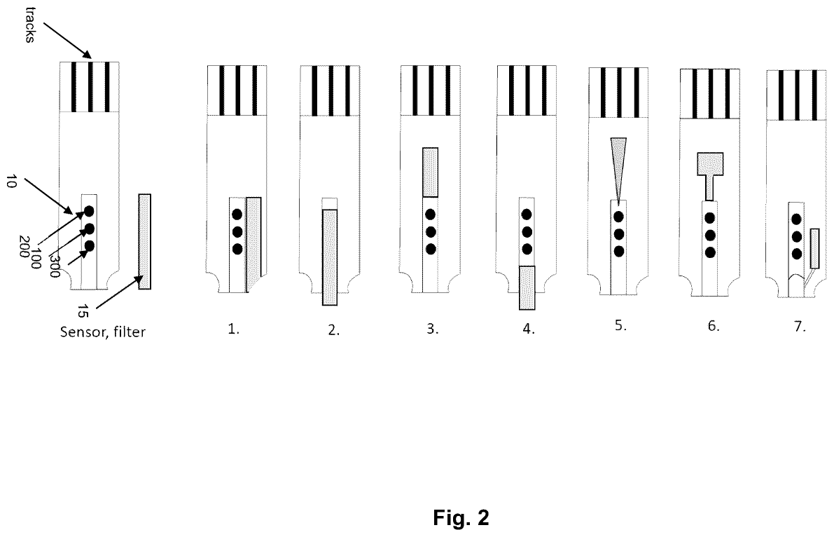

FIG. 2 schematically depicts seven sensors (1 to 7) according to exemplary embodiments, summarized in Table 0.

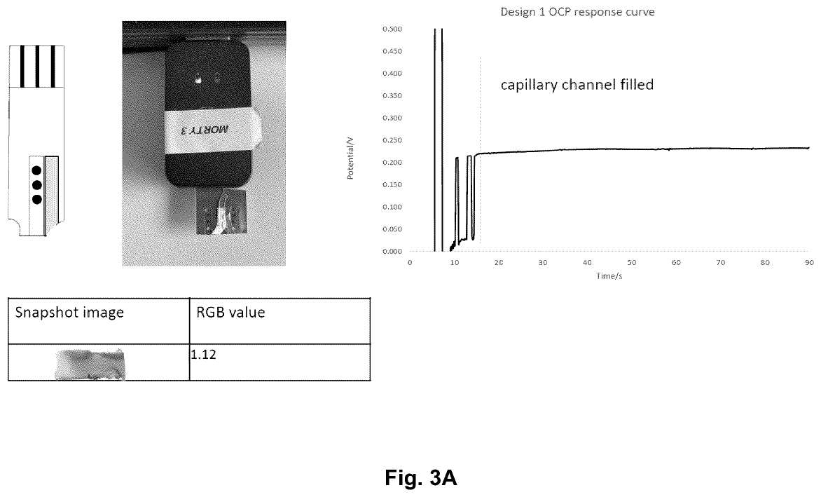

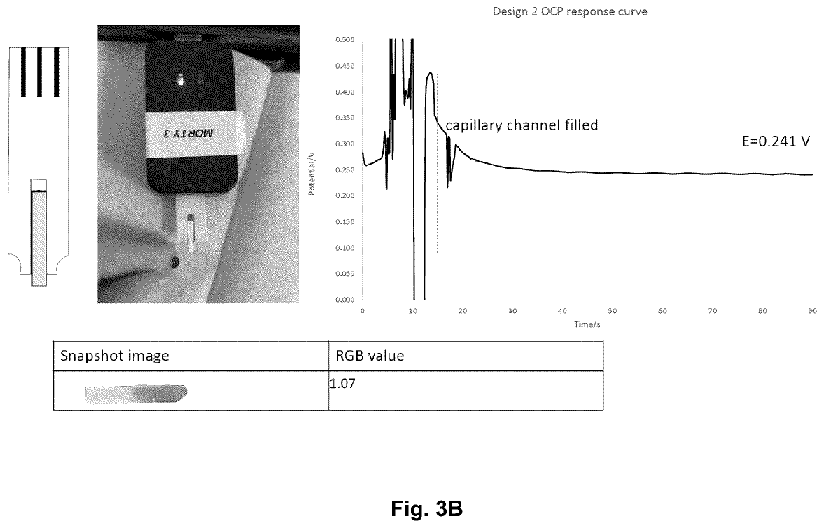

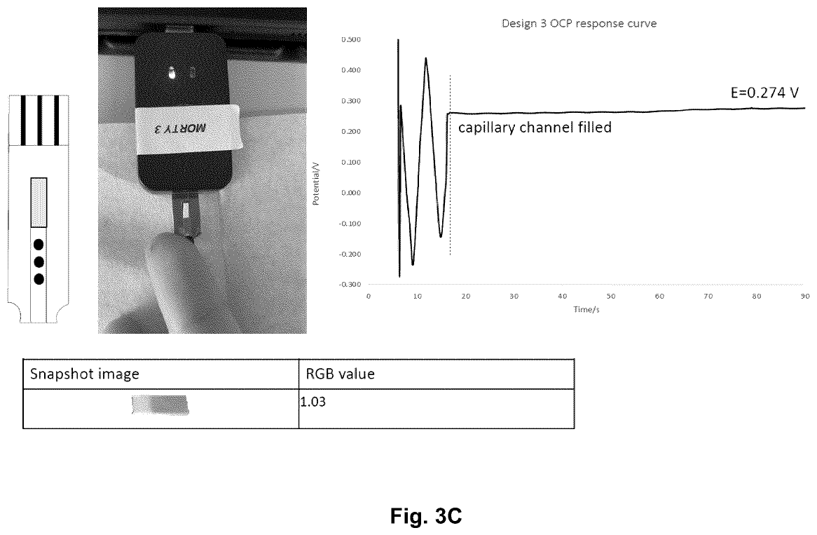

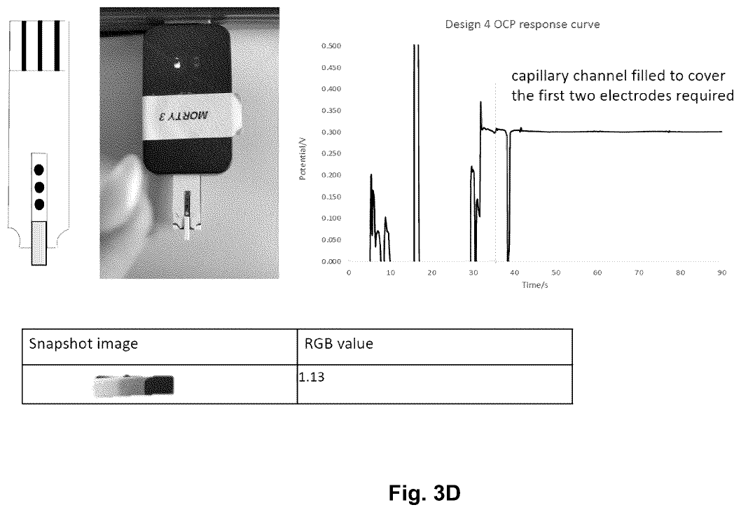

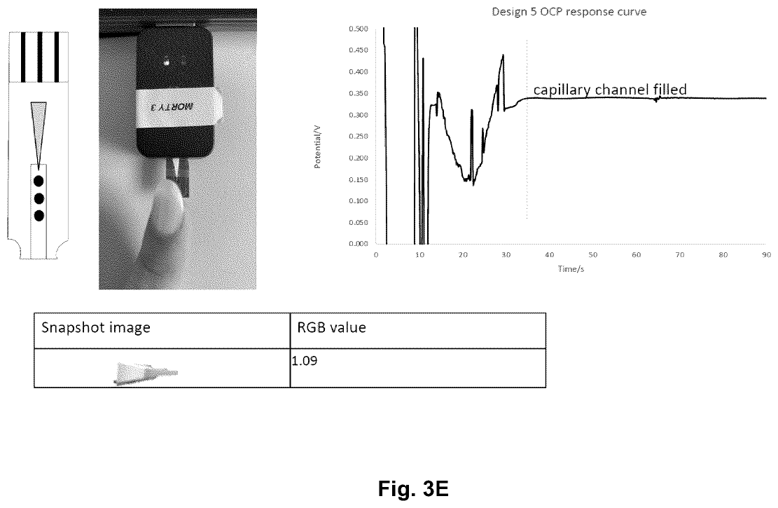

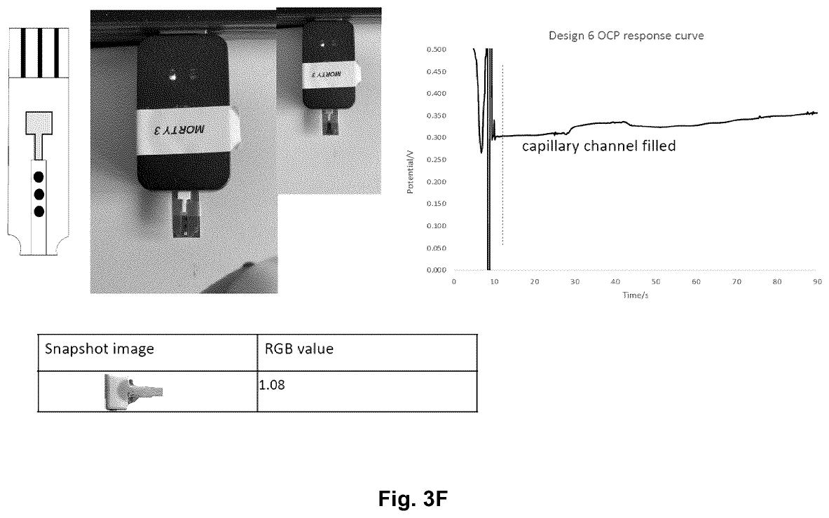

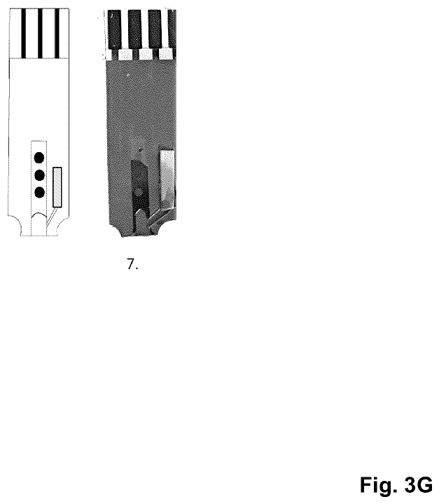

The RGB ratio method of detection of haemolysis on a blood separation filter was used for haemolysis detection during simultaneous detection of potassium on the Kalium Health ion-selective electrode sensor. Seven potential designs are suggested by the author in this paper (FIG. 2). The designs were embedded, and the Open Circuit Potential was displayed over 90 seconds (FIGS. 3A to 3G) for 6 of the designs.

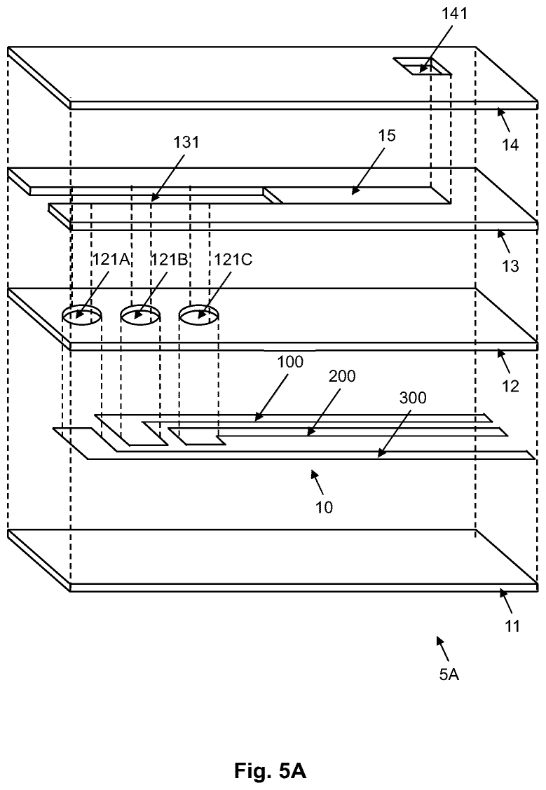

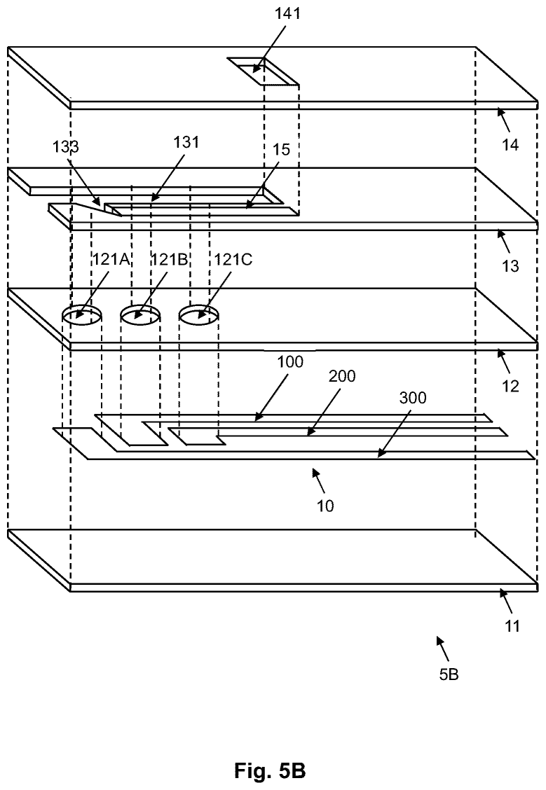

TABLE 0
Seven sensors (1 to 7) according to exemplary embodiments, as shown
in FIG. 2, generally as described with respect to FIGS. 5A and
5B wherein similar reference signs denote like features. Analyte
sensor (potassium ion-selective sensors) and separation device
(Vivid ™ Plasma Separation Membrane (Pall)) as described herein.

| Sensor | Analyte sensor and separation device |
|---|---|
| 1 | The analyte sensor and the separation device |
| | are fluidically coupled mutually in parallel, |
| | wherein the separation device is disposed |
| | beside (i.e. adjacent to) the analyte sensor. |
| 2 | The analyte sensor and the separation device |
| | are fluidically coupled mutually in parallel, |
| | wherein the separation device is disposed |
| | above the analyte sensor. |
| 3 | The analyte sensor and the separation device |
| | are fluidically coupled mutually in series and |
| | the separation device is disposed |
| | downstream of the analyte sensor. |
| 4 | The analyte sensor and the separation device |
| | are fluidically coupled mutually in series and |
| | the separation device is disposed upstream |
| | of the analyte sensor. |
| 5 | The analyte sensor and the separation device |
| | are fluidically coupled mutually in series and |
| | the separation device is disposed |
| | downstream of the analyte sensor. The |
| | capillary bed such as a porous microfilter is |
| | elongate, having a symmetric shape, |
| | particularly a triangle. |
| 6 | The analyte sensor and the separation device |
| | are fluidically coupled mutually in series and |
| | the separation device is disposed |
| | downstream of the analyte sensor. The |
| | capillary bed such as a porous microfilter is |
| | elongate, having a symmetric shape, |
| | particularly a hammer shape. |
| 7 | The analyte sensor and the separation device |
| | are fluidically coupled mutually in parallel, |
| | wherein the separation device is disposed |
| | beside (i.e. adjacent to) the analyte sensor. |
| | See FIG. 5B. |

FIGS. 3A to 3F show open circuit potential (OCP) graphs of potential (V) as a function of time for the six sensors (1 to 6), respectively, of FIG. 2, together with representative images of the separated blood and RGB values of the separated plasma, for potassium measurements of fingerstick blood using an ion-selective electrode, in conjunction to simultaneous haemolysis measurement on filter.

Video recording was the visual recording method selected during this experiment. Photography using a smartphone of the filter proved effective in displaying the clear separation between plasma and red blood cells on the filter while video using a smartphone appears less effective as the plasma is not easy to recognise by eye in the screenshots in FIGS. 3A to 3F and Table 1.

TABLE 1
Results from haemolysis analysis using haemolysis
filter detection method of snapshots of the videos
recorded of sensors 1 to 7 using Equation (2).

| | RGB ratio |
|---|---|
| Sensor | Equation (2) |
| 1 | 1.12 |
| 2 | 1.07 |
| 3 | 1.03 |
| 4 | 1.13 |
| 5 | 1.09 |
| 6 | 1.08 |
| 7 | 1.22 |

The RGB ratio values shown in Table 1 are reasonable, but there is no way to truly validate them as fingerstick blood was used. The volume of the fingerstick draw is insufficient to undergo centrifugation and plasma separation and therefore calculate fHb, as performed previously. The fingerstick blood was also extracted using a very small lancet size (in order to allow the scientist to inflict as many fingerstick punctures as needed with minimum pain) and blood flow was encouraged by squeezing and applying pressure near the opening in order to promote increased blood flow. This is recognised to cause some haemolysis of the fingerstick blood, so as expected different RGB ratios were recorded for these measurements.

Sensors used were of different age past ion-selective functionalisation and therefore may provide a different OCP response to potassium in blood for each repeat. Furthermore, these seven repeats were carried out on different days and conditions were not recorded or optimised in order to produce a reliable potassium reading. Potassium was not monitored for.

This was because the particular aim of this experiments was to assess the best way to embed the filter in order not to disturb the measurement of potassium (straight line horizontal response of the potential vs time graph). Design 1 and 4 performed extremely well and the capillary channel and filter filled easily whilst maintaining undisturbed OCP (FIG. 2). Design 6 performed poorly due to most of the blood in the channel being drawn into the filter and causing disturbances in the response at t=30 seconds (FIG. 3). Similarly Design 3 and 5 may face similar challenges. Design 2 may be a potential candidate if equilibration time is allowed for the potential to stabilise overtime. Although some designs may appear more successful than others all design could be modified further in order to achieve better outcomes. For example, Design 3 could be modified to include a filter much smaller in size in order to circumvent large volumes of blood to be drawn away from electrode. The rapidity of the quality control feature is also of importance (2-10 seconds depending on strip size). The filter separation of plasma and blood occurs at the same time as the filling of the capillary channel of the sensor and the data recording.

The experiment may be further improved by using a photograph as the method of recording, instead of video. The quality of the camera may also provide improvements to the results. A device, where the setup of camera and lighting is standardised would be devised in order to minimise error due to poor colour recording.

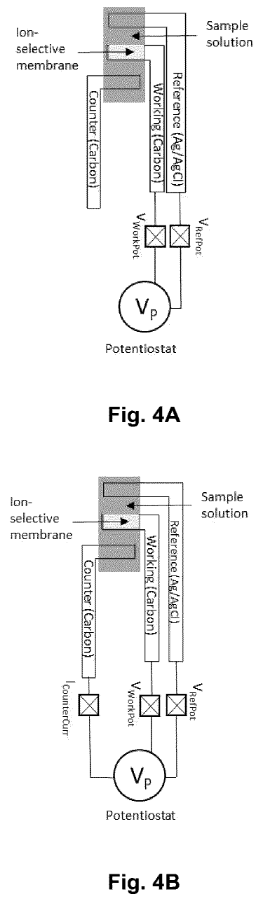

FIG. 4A schematically depicts an ion-selective electrode cell for a sensor according to an exemplary embodiment. Particularly, the ion-selective electrode cell comprises the ISE and the RE (i.e. a two electrode ion-selective electrode cell), as described above, and is depicted in a circuit with a potentiostat. Generally, a potentiostat is electronic hardware required to control a three electrode cell and run most electroanalytical experiments. A bipotentiostat and a polypotentiostat are potentiostats capable of controlling two working electrodes and more than two working electrodes, respectively. The potentiostat functions by maintaining the potential of the working electrode at a constant level with respect to the reference electrode by adjusting the current at an auxiliary electrode. It consists of an electric circuit which is usually described in terms of simple op amps.

FIG. 4B schematically depicts an ion-selective electrode cell for a sensor according to an exemplary embodiment. Particularly, the ion-selective electrode cell comprises the ISE, the RE and the CE (i.e. a three electrode ion-selective electrode cell), as described above, and is depicted in a circuit with a potentiostat.

FIG. 5A schematically depicts an exploded, perspective view of a sensor 5A according to an exemplary embodiment. The sensor 5A is similar to the sensor 3 of FIG. 2, FIG. 3C and Table 1.

The sensor is for sensing analytes in blood comprising plasma and erythrocytes. The sensor comprises an analyte sensor 10 configured to sense an analyte in the blood and to output a response corresponding to the sensed analyte and a separation device 15 adapted to mutually separate the plasma and the erythrocytes, for assessing a haemolysis level of the blood based on a colour of the separated plasma.

In this example, the analyte sensor 10 and the separation device 15 are fluidically coupled mutually in series.

In this example, the separation device 15 is a passive separation device, based on a porous microfilter. In this example, the porous microfilter is Vivid™ Plasma Separation Membrane.

In this example, the porous microfilter is elongate, having a symmetric shape, particularly a rectangle.

In this example, the separation device 15 is disposed for assessing the haemolysis level of the blood based on the colour of the separated plasma measured by reflectance.

In this example, a surface of the separation device 15 is exposed, via an aperture 141.

In this example, the analyte sensor 10 is an ion-selective electrode cell, comprising an ion-selective electrode 100, a reference electrode 200 and a counter electrode 300.

In this example, the ion-selective electrode 100, the reference electrode 200 and the counter electrode 300 are disposed in and/or on the separation device 15.

In this example, the ion-selective electrode cell 10 comprises the ISE 100 and a reference electrode, RE, 200, together with a counter electrode, CE, 300 provided by screen printing on a substrate layer 11. Each electrode is L-shaped, including a track, provided by the long leg of the L, extending to a first end of the substrate layer 11 for coupling to a potentiostat, for example.

Three corresponding circular apertures 121A, 121B, 121C are provided in a mask layer 12 that overlays the ISE 100, the RE 200 and the CE 300, thereby revealing circular portions of these respective electrodes, particularly in the short leg of the respective L. A channel layer 13 overlays the mask layer 12, having therein a channel 131 that extends from a second end of the channel layer 13, distal to the first end of the separation device 15, towards an opposed first end of the channel layer 13, whereby the circular apertures 121A, 121B, 121C are fully within the channel 131. A cover layer 14 overlays the channel layer 13 and includes a square aperture 141 coincident with the end of the separation device 15.

In this example, the separation device 15 is provided on the mask layer 12, in the channel 131. FIG. 5B schematically depicts an exploded, perspective view of a sensor 5B according to an exemplary embodiment. The sensor 5B is generally as described with respect to the sensor 5A, description of which is not repeated for brevity. The sensor 5A is similar to the sensor 7 of FIG. 2 and FIG. 3G.

In this example, the analyte sensor 10 and the separation device 15 are fluidically coupled mutually in parallel, via a bifurcation 133 of the channel 131 in the channel layer 13.

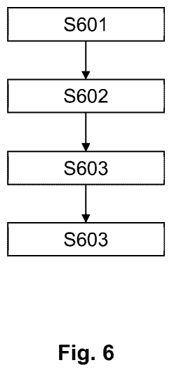

FIG. 6 schematically depicts a method according to an exemplary embodiment.

The method is of sensing analytes in blood comprising plasma and erythrocytes. The method comprises:
mutually separating the plasma and the erythrocytes (S601);
sensing an analyte in the blood and/or the plasma and outputting a response corresponding to the sensed analyte (S602);
assessing a haemolysis level of the blood based on a colour of the separated plasma (S603); and indicating a quality of the response corresponding to the sensed analyte based on the haemolysis level (S604).

The method may include any of the steps described herein.

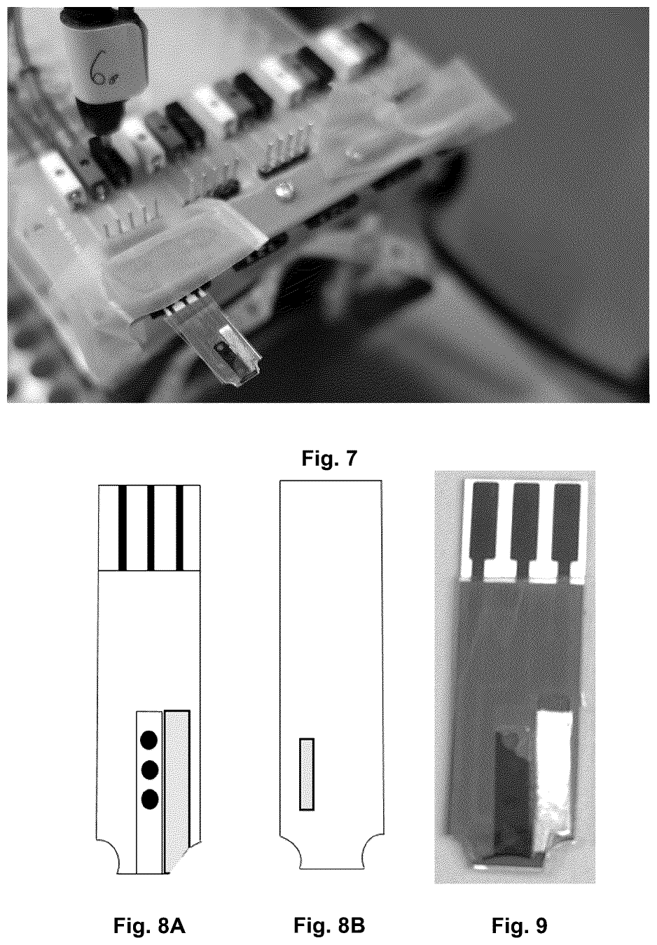

FIG. 7 is a photograph of an apparatus according to an exemplary embodiment.

The method is generally as described previously. Vivid™ Plasma Separation Membrane (Pall) GX was used as a lateral separator of plasma from whole blood. Potassium ion-selective sensors used were created by Sanwald GmbH Technischer Siebdruck and functionalised by Kalium Health. Capillary channels were prepared by Quick Axis Ltd for Kalium Health. Venous blood was obtained from volunteer and a lithium-heparin S-Monovette 4.9 mL was used to store venous blood. The blood was centrifuged using an Eppendorf 5415 D. Fingerpick blood obtained from scientist performing experiments using a Haemolance plus 21 G 1.8 mm lancet. Spectrophotometer used was the NeoDot UV/Vis Nano Spectrophotometer (Generon). Potentiostat used was the MultiPalmSens4 (Palm Sens) connected to a custom printed circuit board interface. Images and videos were captured using the Sony Cyber-shot DSC-RX100 Va Camera, 4K, 20.1 MP. Potassium concentration in blood samples was measured using i-STAT1 (Abbott Point of Care Inc.) following manufacturer instructions.

FIG. 8A is a plan view from above of a sensor according to an exemplary embodiment; and FIG. 8B is a plan view from below of the sensor. FIG. 9 is a photograph from above of a sensor according to an exemplary embodiment. These sensors are generally as described with respect to FIG. 2 sensor 1 and FIG. 3A.

Filter embedding, electrochemical measurement and RGB ratio measurement: The blood separating filter was embedded onto the Kalium Health sensors in a parallel configuration, placed on top of an opening in the sensor. A 10 μL volume (overfill) was placed on the lip of the opening shared by both the sensor channel and filter. The Open Circuit Potential (OCP) between the ion-selective working electrode and the reference electrode was recorded over 90 seconds, t interval=0.2 measuring the response of the sensor to potassium in whole blood, whilst simultaneously recording video of the filter separating the whole blood. The protocol for measuring fHb is as described previously.

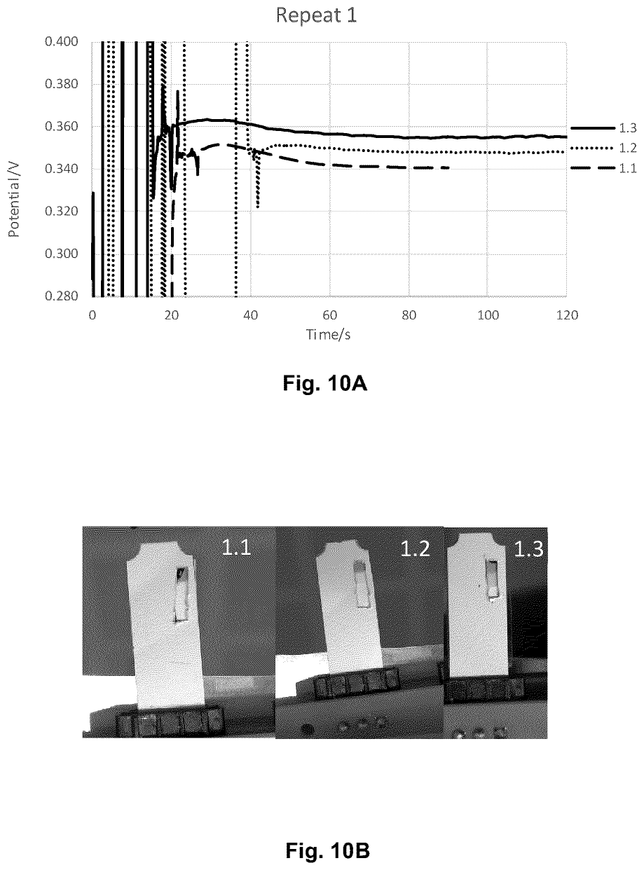

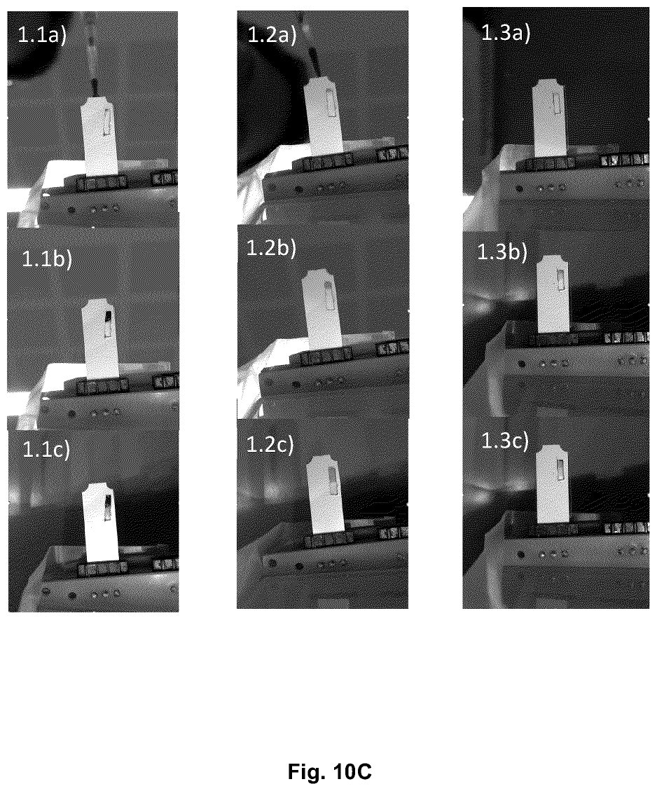

FIG. 10A is a graph of OCP response against time for each blood sample, recorded alongside the haemolysis detection device (Measurement begins before blood is applied. As blood is applied, spikes in response may be observed. The evolution of peaks in the curve is due to equilibration. OCP value for each curve recorded once response is stable); FIG. 10B shows images taken from videos recorded during the simultaneous potassium detection measurement and filter filling with blood—these images were analysed to produce the RGB values displayed; and FIG. 10C shows images from videos recorded during sensor and filter filling with blood—OCP was recorded simultaneously.

TABLE 2
Potassium concentration, fHb, RGB ration
and OCP for sample 1 (repeat 1) (n = 3).

| | | | | OCP/V |
|---|---|---|---|---|
| | | Free | RGB ratio | (Measured |
| | | haemoglobin | Equation (2) | using |
| | Potassium | fHb | (Measured | exemplary |
| | Concen- | (gL⁻¹) | using | sensors) |
| | tration | (Measured | exemplary | (calibration |
| | in blood (mM) | using | sensors using | required for |
| Sample | (Measured | Spectropho- | exemplary | conversion |
| number | using iStat) | tometer) | method) | to mM) |
| 1.1 | 4.0 | 0.027 | 0.83 | 0.341 |
| 1.2 | 4.7 | 0.18 | 0.92 | 0.348 |
| 1.3 | 5.7 | 0.59 | 1.09 | 0.355 |

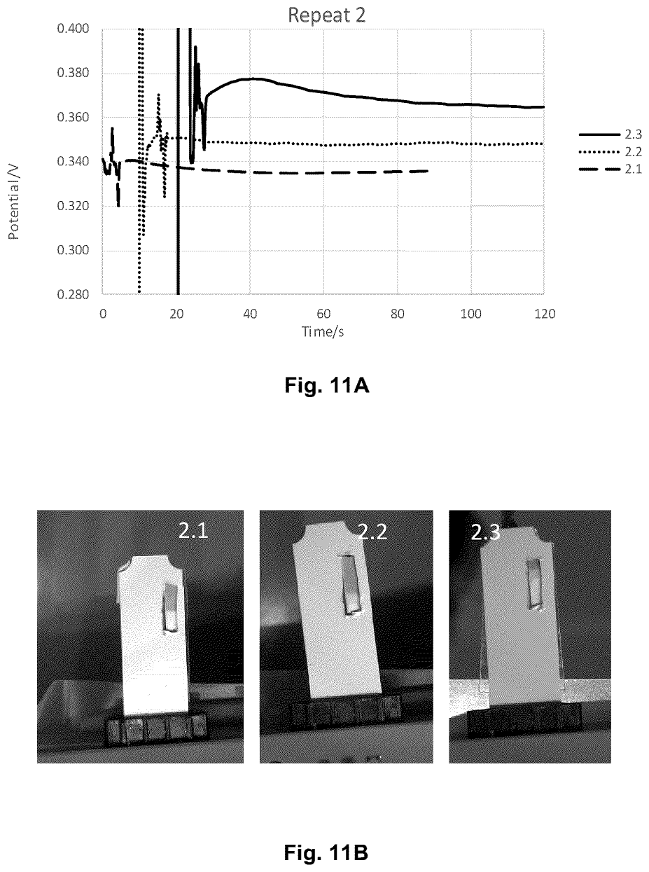

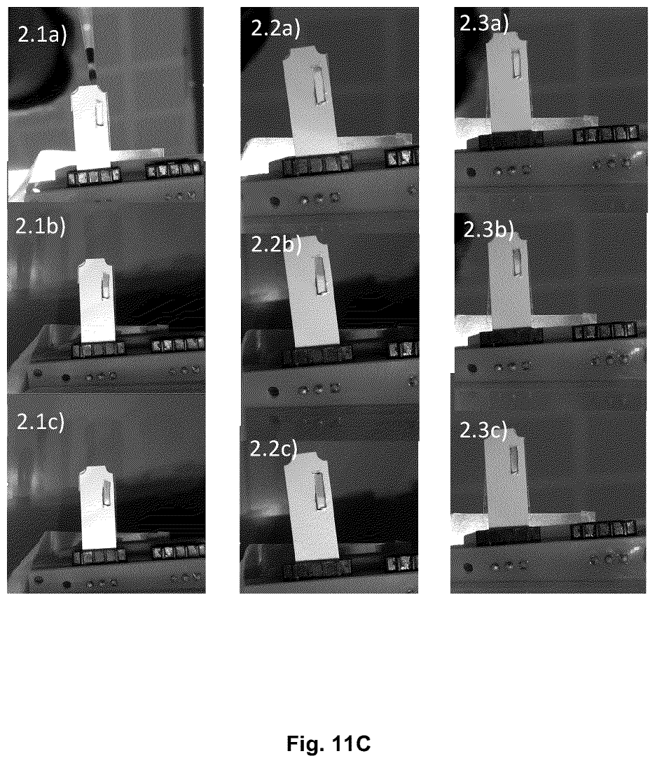

FIG. 11A is a graph of OCP response against time for each blood sample, recorded alongside the haemolysis detection device; FIG. 11B shows images taken from videos recorded during the simultaneous potassium detection measurement and filter filling with blood—these images were analysed to produce the RGB values displayed; and FIG. 11C shows images from videos recorded during sensor and filter filling with blood—OCP was recorded simultaneously.

TABLE 2
Potassium concentration, fHb, RGB ration
and OCP for sample 1 (repeat 2) (n = 3).

| | | | | OCP/V |
|---|---|---|---|---|
| | | Free | RGB ratio | (Measured |
| | | haemoglobin | Equation (2) | using |
| | Potassium | fHb | (Measured | exemplary |
| | Concen- | (gL⁻¹) | using | sensors) |
| | tration | (Measured | exemplary | (calibration |
| | in blood (mM) | using | sensors using | required for |
| Sample | (Measured | Spectropho- | exemplary | conversion |
| number | using iStat) | tometer) | method) | to mM) |
| 2.1 | 4.0 | 0.027 | 0.86 | 0.335 |
| 2.2 | 4.7 | 0.18 | 0.98 | 0.347 |
| 2.3 | 5.7 | 0.59 | 1.02 | 0.364 |

Standard method used to obtain haemolysis levels of samples:
1. Venous blood is obtained from volunteer;
2. Blood is centrifuged to separate red blood cells and plasma;
3. 1.2 μL volume of plasma is applied on the spectrophotometer stage of the Neodot Spectrophotometer in order to calculate free haemoglobin from spectra.

How free haemoglobin concentration fHb calculated:
1. Using the Neodot Spectrophotometer (blanked using extra pure water) 190-1100 nm range, the Absorbance at 380 nm, 415 nm and 450 nm values were obtained;
2. fHb calculated using Equation (1).

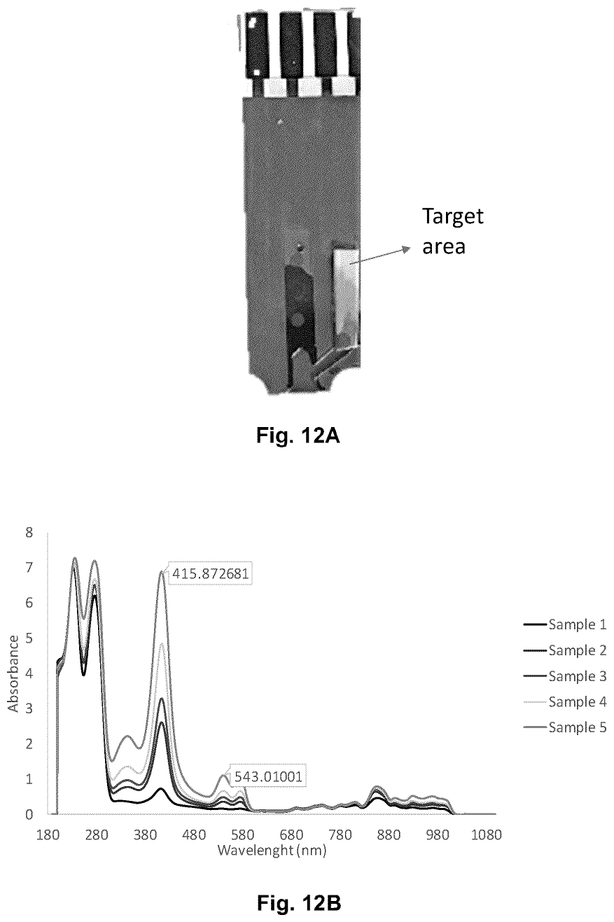

FIG. 12A is a photograph from above of a sensor according to an exemplary embodiment; and FIG. 12B is a graph of absorbance as a function of wavelength showing raw spectra of extracted plasma samples 1 to 5 (increasing haemolysis levels) between 190-1100 nm measured using NeoDot UV/Vis Nano Spectrophotometer.

Sample 1 has the lowest haemolysis level (having the lowest absorbance at 415 nm) and Sample 5 has the highest haemolysis level (having the highest absorbance at 415 nm).

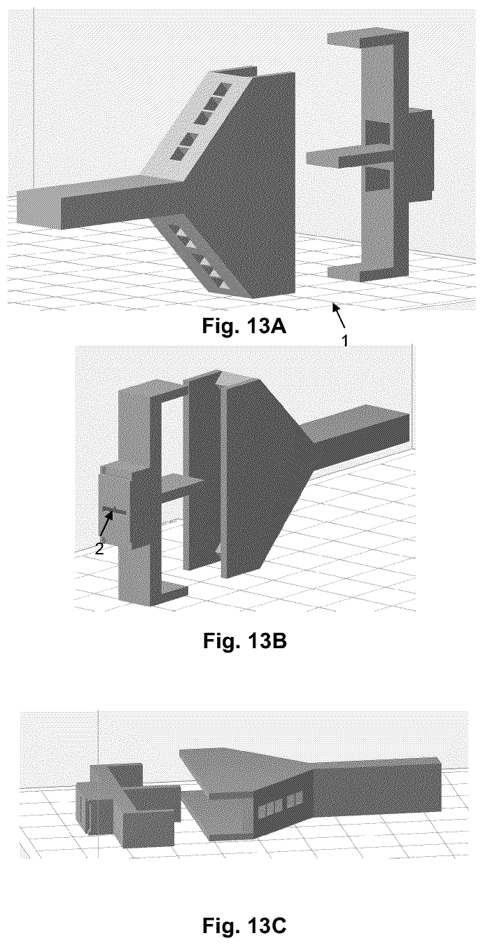

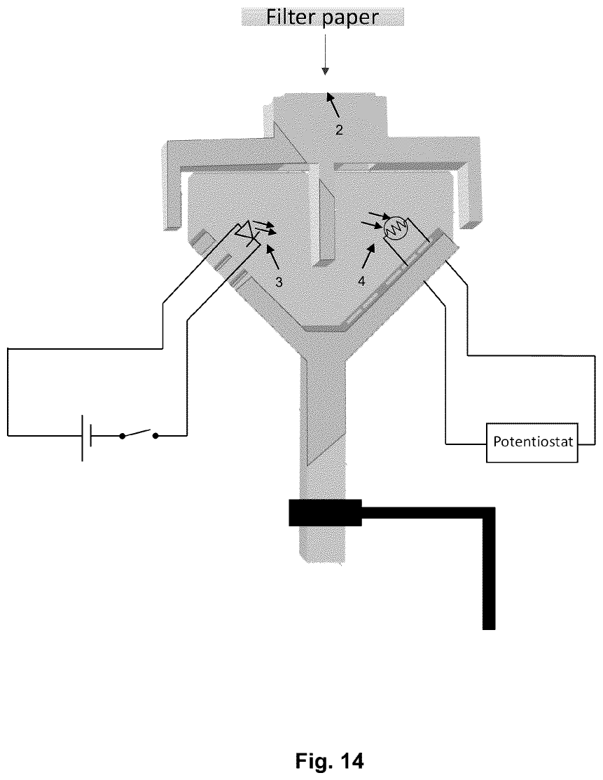

FIGS. 13A to 13C are 3D printed structure of an apparatus 1 according to an exemplary embodiment, showing LED 3, slit 2 and photoresistor 4. FIG. 14 is a sectional view of the 3D printed structure of FIGS. 13A to 13C, secured by a clamp stand of the device and the installation positions of the LED and photoresistor, in more detail.

Filter paper (i.e. separation device) placed on top of apparatus 1 and blood applied to one end of the filter paper. Light from LED 3 is incident on the filter paper via the slit 2. Light reflected from the filter paper via the slit 2 is detected by photoresistor 4. The filter paper may be placed in contact with the apparatus 1, over the slit 2. The fixed positions of the LED 3, slit 2 and photoresistor 4 determine the angles and distances of incidence and reflection, improving quantification of absorbance and/or repeatability of measurement. LED 3: Green 540 nm; ASM6-SG91-NRTOH Green; Wavelength: Min 515, Typ 525, Max 535 nm. LDR 4: Silonex, Through Hole LDR (Light Dependent Resistor) 7000 Light, 0.67 MΩ Dark, 2-Pin TO-18. Emstat Pico: Chronoampterometry measurement (2V, 0.2 s)

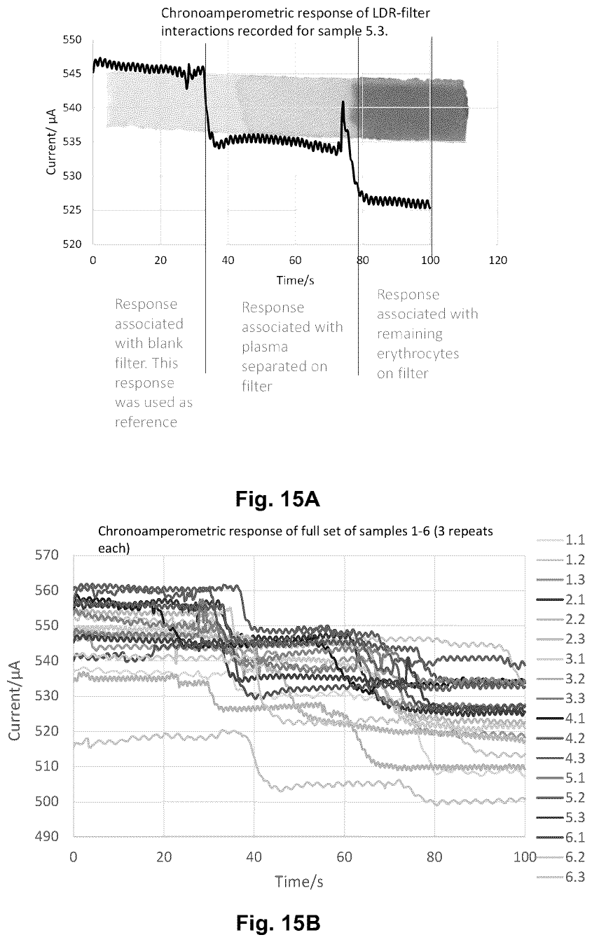

FIG. 15A is a graph of chronoamperometric response of LDR-filter interactions recorded for sample 5.3, overlaying a photograph of the filter of the sample 5.3; and FIG. 15B is a graph of chronoamperometric response of full set of samples 1 to 6 (3 repeats each).

TABLE 4
Information about samples 1 to 6, assessing their haemolysis
and the absorbance at wavelengths 380 nm-450 nm required
for the calculation of free haemoglobin fHb (g/L).

| Sample | Visually | Abs. at | Abs. at | Abs. at | fHb (g/L) |
|---|---|---|---|---|---|
| number | haemolysed? | 380 nm | 415 nm | 450 nm | Equation (1) |
| 1 | Yes | 0.530327 | 1.276431 | 0.406705 | 0.135 |
| 2 | Yes | 0.869865 | 2.323142 | 0.623814 | 0.260 |
| 3 | Yes | 0.718129 | 2.517373 | 0.470881 | 0.320 |
| 4 | Yes | 1.043322 | 3.714777 | 0.675001 | 0.477 |
| 5 | Yes | 1.840055 | 5.954316 | 1.088398 | 0.750 |
| 6 | Yes | 1.126465 | 4.198436 | 0.698057 | 0.549 |

Change in current is given by Equation (5):

$$Δ i = {i}_{blank} - {i}_{plasma} \qquad Equation ( 5 )$$

TABLE 5
Change in current described by Equation 2X
for samples 1-6 (3 repeats per sample).

| Sample number.Repeat number | Δi |
|---|---|
| 1.1 | 6.698 |
| 1.2 | 5.994 |
| 1.3 | 4.985 |
| 2.1 | 9.424 |
| 2.2 | 8.284 |
| 2.3 | 11.629 |
| 3.1 | 9.568 |
| 3.2 | 9.099 |
| 3.3 | 8.895 |
| 4.1 | 11.659 |
| 4.2 | 10.922 |
| 4.3 | 12.206 |
| 5.1 | 9.039 |
| 5.2 | 14.177 |
| 5.3 | 10.736 |
| 6.1 | 11.804 |
| 6.2 | 12.591 |
| 6.3 | 18.626 |

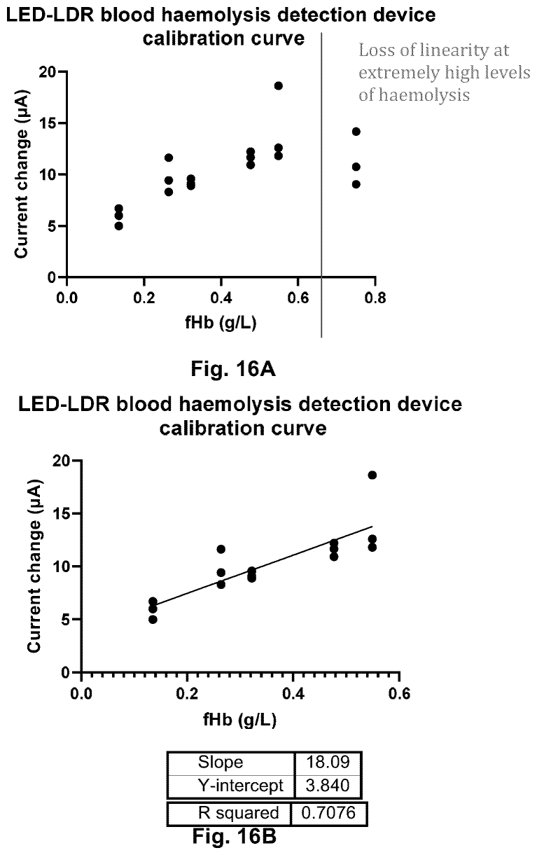

FIG. 16A is a graph of current change as a function of fHb for the full set of samples 1 to 6 (3 repeats each); and FIG. 16B is a graph of current change as a function of fHb for the full set of samples 1 to 6 (3 repeats each), excluding those samples in the non-linear region. Slope=19.09; y intercept=3.840; R²=0.7076.

In summary, the novel sensor, apparatus, kit and method provided assessment of haemolysis of blood sample via an easy to automate reading of visual appearance of plasma on a lateral blood separation filter. The novel RGB ratio method described compares well to the well-established plasma/serum haemoglobin measurement used in literature. Different designs of embedding the filter-based detection method alongside a potassium ion-selective sensor were explored. The technique proved promising due to the speed of the detection, the ease of the data processing, the low price of the equipment required, and the extremely low volume of blood needed (a few NL). Moreover, this technique has the potential to aid not only alongside POC sensing, but in lab setting, as it requires no training (such as centrifugation of blood) in order to perform.

Although a preferred embodiment has been shown and described, it will be appreciated by those skilled in the art that various changes and modifications might be made without departing from the scope of the invention, as defined in the appended claims and as described above.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims and drawings), and/or all of the steps of any method or process so disclosed, may be combined in any combination, except combinations where at most some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims and drawings), or to any novel one, or any novel combination, of the steps of any method or process so disclosed.

### REFERENCES

1 L. Heireman, P. van Geel, L. Musger, E. Heylen, W. Uyttenbroeck and B. Mahieu, Clinical Biochemistry, 2017, 50, 1317-1322.
2 E. J. Mccaughey, E. Vecellio, R. Lake, L. Li, L. Burnett, D. Chesher, S. Braye, M. Mackay, S. Gay, T. C. Badrick, J. I. Westbrook and A. Georgiou, Royal College of Pathologists Australasia Quality Assurance Program.
3 D. Monneret, F. Mestari, G. Atlan, C. Corlouer, Z. Ramani, J. Jaffre, S. Dever, V. Fressart, R. Alkouri, F. Lamari, C. Devilliers, F. Imbert-Bismut and D. Bonnefont-Rousselot, Scandinavian Journal of Clinical and Laboratory Investigation, 2015, 75, 162-169.
4 E. Aloisio, A. Carnevale, S. Pasqualetti, S. Birindelli, A. Dolci and M. Panteghini, Clinical Biochemistry, 2018, 57, 62-64.
5 S. O. Sowemimo-Coker, Transfusion Medicine Reviews, 2002, 16, 46-60.
6 N. M. Denihan, B. H. Walsh, S. N. Reinke, B. D. Sykes, R. Mandal, D. S. Wishart, D. I. Broadhurst, G. B. Boylan and D. M. Murray, Clinical Biochemistry, 2015, 48, 534-537.
7 J. Ni, W. Zhu, Y. Wang, X. Wei, J. Li, L. Peng, K. Zhang and B. Bai, Journal of Clinical Laboratory Analysis, DOI:10.1002/JCLA.23561.
8 C. W. Weng, B. C. Hsieh, Y. te Hou and T. J. Cheng, Analyst, 2015, 140, 6619-6624.
9 T. van Buren, G. Arwatz and A. J. Smits, Scientific Reports 2020 10:1, 2020, 10, 1-5.
10 C. Kollegger, P. Greiner, I. Siegl, C. Steffan, M. Wiessflecker, B. Pekec, M. Hajnsek, F. Sinner, G. Holweg and B. Deutschmann, Elektrotechnik und Informationstechnik, 2018, 135, 83-88.
11 C. Zhou, M. Keshavarz Hedayati, X. Zhu, F. Nielsen, U. Levy and A. Kristensen, ACS Sensors, 2018, 3, 784-791.
12 M. Bond, C. Elguea, J. S. Yan, M. Pawlowski, J. Williams, A. Wahed, M. Oden, T. S. Tkaczyk and R. Richards-Kortum, Lab on a Chip, 2013, 13, 2381-2388.
13 X. Yang, N. Z. Piety, S. M. Vignes, M. S. Benton, J. Kanter and S. S. Shevkoplyas, Clinical Chemistry, 2013, 59, 1506-1513.
14 Vivid™ Plasma Separation Membrane—Diagnostics, https://shop.pall.co.uk/uk/en/medical/advanced-materials/diagnostics-2/zidgri78lls, (accessed Apr. 22, 2022).
15 W. Guo, J. Hansson and W. van der Wijngaart, Analytical Chemistry, 2020, 92, 6194-6199.
16 R. H. Tang, H. Yang, J. R. Choi, Y. Gong, S. S. Feng, B. Pingguan-Murphy, Q. S. Huang, J. L. Shi, Q. B. Mei and F. Xu, https://doi.org/10.3109/07388551.2016.1164664, 2016, 37, 411-428.
17 H. Li and A. J. Steckl, Analytical Chemistry, 2019, 91, 352-371.

1. A sensor for sensing analytes in blood comprising plasma and erythrocytes, the sensor comprising:
an analyte sensor configured to sense an analyte in the blood and to output a response corresponding to the sensed analyte; and
a separation device adapted to mutually separate the plasma and the erythrocytes, for assessing a haemolysis level of the blood based on a colour and/or a fluorescence of the separated plasma.

2. The sensor according to claim 1, wherein the analyte sensor and the separation device are fluidically coupled mutually in parallel.

3. The sensor according to claim 1, wherein the analyte sensor and the separation device are fluidically coupled mutually in series.

4. The sensor according to claim 3, wherein the separation device is disposed downstream or upstream of the analyte sensor.

5. The sensor according to claim 1, wherein the separation device comprises and/or is an active separation device, for example based on bifurcation, constriction-expansion, biophysical and/or geometrical separation, and/or a passive separation device, for example based on a microbead plug, a capillary bed such as a porous microfilter, a microstructured polymer or a sintered polymer, a membrane filter, blood agglutination, a nanofilter and/or superhydrophobicity.

6. The sensor according to claim 5, wherein the separation device comprises and/or is a passive separation device based on a capillary bed such as a porous microfilter, a microstructured polymer or a sintered polymer.

7. The sensor according to claim 6, wherein the capillary bed is elongate, having a symmetric shape such as a rectangle, a triangle or a hammer shape.

8. The sensor according to claim 1, wherein the separation device is disposed for assessing the haemolysis level of the blood based on the colour and/or the fluorescence of the separated plasma measured optically, for example by absorbance, reflectance and/or transmission.

9. The sensor according to claim 1, wherein a surface of the separation device is exposed, for example directly or indirectly exposed such as to the ambient for example via an aperture and/or optically exposed for example via an optically transparent window.

10. The sensor according to claim 1, wherein the analyte sensor comprises and/or is an ion-selective electrode cell, comprising an ion-selective electrode, a reference electrode and optionally, a counter electrode.

11. (canceled)

12. The sensor according to claim 1, wherein the analyte is one or more selected from the group consisting of potassium (K), lactate dehydrogenase (LDH), aspartate aminotransferase (AST), alanine aminotransferase, magnesium, phosphate, folate, urea, glucose, sodium, chloride, bilirubin, gamma-glutamyltransferase, alkaline phosphatase, albumin, insulin, glucagon, calcitonin, parathyroid hormone, adrenocorticotropic hormone, gastrin, alkaline phosphatase, gamma-glutamyltransferase, bilirubin, creatine kinase and troponin T.

13. An apparatus configured to receive a sensor according to claim 1, comprising:
an analyte detector configured to detect the response, corresponding to the sensed analyte, output from the analyte sensor; and
an optical detector configured to detect the colour and/or the fluorescence of the separated plasma for assessing the haemolysis level of the blood.

14. The apparatus according to claim 13, configured to output a value corresponding with the haemolysis level of the blood based on the detected colour and/or the detected fluorescence of the separated plasma.

15. (canceled)

16. The apparatus according to claim 14, configured to implement a remedial action based on the value.

17. A kit comprising:
a sensor, wherein the sensor comprises:
an analyte sensor configured to sense an analyte in the blood and to output a response corresponding to the sensed analyte, and
a separation device adapted to mutually separate the plasma and the erythrocytes, for assessing a haemolysis level of the blood based on a colour and/or a fluorescence of the separated plasma; and
an apparatus according to claim 13.

18. A method of sensing analytes in blood comprising plasma and erythrocytes, the method comprising:
mutually separating the plasma and the erythrocytes;
sensing an analyte in the blood and/or the plasma and outputting a response corresponding to the sensed analyte;
assessing a haemolysis level of the blood based on a colour and/or a fluorescence of the separated plasma; and
indicating a quality of the response corresponding to the sensed analyte based on the haemolysis level.

19. The sensor according to claim 10, further comprising
a substrate layer on which each of the ion-selective electrode, the reference electrode and the counter electrode are printed and which extend to a first end of the substrate layer for coupling to a potentiostat,
a mask layer which overlays each of the ion-selective electrode, the reference electrode and the counter electrode and which has three apertures, one above each electrode, and
a channel layer which overlays the mask layer, wherein the channel layer comprises the separation device and a channel in which the apertures are located.

20. The sensor according to claim 19, wherein each electrode is L-shaped and the long leg of the L extends to a first end of the substrate layer and the apertures in the mask layer are above the short legs of the respective L.

21. The sensor according to claim 19, further comprising a cover layer which overlays the channel layer and includes an aperture coincident with an end of the separation device.

22. The apparatus according to claim 14 wherein the apparatus is configured to output a value corresponding with the haemolysis level of the blood based on the detected colour of the separated plasma, by
determining a red index, a green index and a blue index for the detected colour of the separated plasma,
calculating a ratio of the red index to one of the green index, the blue index and an average of the green index and blue index, and
outputting the calculated ratio as a value corresponding with the haemolysis level.
